(19) United States
(12) Patent Application Publication
(10) Pub. No.: US 2023/0314443 A1
(43) Pub. Date: Oct. 5, 2023

(54) METHODS OF IDENTIFYING INTERACTIONS OF A COMPOUND AND A CONDENSATE, OR A COMPONENT THEREOF, AND USES THEREOF

(71) Applicant: Dewpoint Therapeutics, Inc., Boston, MA (US)

(72) Inventors: Diana Maria MITREA, Boston, MA (US); Matthäus MITTASCH, Dresden (DE); Peter Jeffrey DANDLIKER, Boston, MA (US); Bruce Aaron BEUTEL, Boston, MA (US); Edgar Erik BOCZEK, Dresden (DE)

(21) Appl. No.: 18/020,897

(22) PCT Filed: Aug. 11, 2021
(86) PCT No.: PCT/US2021/045592

§ 371 Date: Feb. 10, 2023

Related U.S. Application Data
(60) Provisional application No. 63/064,867, filed on Aug. 12, 2020.

(51) Int. Cl. G01N 33/68; C12N 15/10

(52) U.S. Cl. CPC G01N 33/6845; C12N 15/1055

(57) ABSTRACT

In some aspects, provided herein are methods of identifying interactions of a compound and a condensate, or a component thereof, and uses thereof. In other aspects, provided herein are methods of identifying (or screening for or designing) compounds, or portions thereof, having a desired interaction with a condensate, or a component thereof. In yet other aspects, provided herein are applications of the methods described herein, e.g., libraries of compounds having known or predicted characteristics, and methods of identifying compounds useful for treatment of a disease.

### CROSS-REFERENCE TO RELATED APPLICATIONS

This application claims priority benefit of U.S. Provisional Application No. 63/064,867, filed on Aug. 12, 2020, the content of which is incorporated herein by reference in its entirety.

### TECHNICAL FIELD

The present application relates to the field of biological condensates.

### BACKGROUND

In addition to membrane-bound organelles, cells and organisms contain distinct features containing molecules not enclosed by a membrane separating the feature from the immediately surrounding solution. These membrane-less molecular assemblies have been shown to be formed through a process termed liquid-liquid phase separation (LLPS) or condensation. The membrane-less feature, e.g., a condensate, forms a dense phase containing a concentration of molecules, such as biological macromolecules including polypeptides and nucleic acids, and is directly surrounded by a non-phase separated light phase. In some instances, condensates are a dynamic cellular feature exhibiting changes in, e.g., presence, composition, physical state, and morphological features over time and under different conditions/stimuli. A number of condensates are well recognized in the art, including both cellular, extra-cellular, and in vitro condensates. See, e.g., Alberti et al., J Mol Biol, 430, 2018, 4806-4820; and Muiznieks et al., J Mol Biol, 430, 2018, 4741-4753.

Condensates are known to be involved in the function of certain cellular processes and may influence, to some degree, the efficacy and safety of certain therapeutic agents. Condensates can bring together certain molecules, including endogenous molecules and exogenous molecules, at an elevated concentration relative to the surrounding light phase. In some instances, the localization of certain molecules may enable and/or accelerate a reaction inside the condensate by brining certain molecules together in close proximity. In some instances, the localization of certain molecules may sequester a certain molecule from the surrounding light phase and, e.g., inhibit the ability of that molecule to act in a certain manner. In the budding field of biological condensates, there is much that is still unknown about mechanisms governing condensate partitioning and the effect of a compound on a condensate, much less techniques for studying the interactions between a compound (or a portion thereof) and a condensate (or a component thereof).

### BRIEF SUMMARY

The present invention in one aspect provides a method of identifying one or more interactions of a test compound, or a portion thereof, and a target condensate, or a component thereof, the method comprising: obtaining two or more of: (i) a partition characteristic of the test compound, or the portion thereof, for the target condensate; (ii) a binding affinity characteristic of the test compound, or the portion thereof, for the component of the target condensate in a light phase; or (iii) a phase boundary characteristic of the component of the target condensate in the presence of the test compound, or the portion thereof; and identifying the one or more interactions of the test compound, or the portion thereof, and the target condensate, or the component thereof, based on comparing two or more of (i), (ii), and (iii) to identify the one or more interactions. In some embodiments, identifying the one or more interactions of the test compound, or the portion thereof, and the target condensate, or the component thereof, is based on comparing the partition characteristic of the test compound, or the portion thereof, for the target condensate; and the binding affinity characteristic of the test compound, or the portion thereof, for the component of the target condensate in the light phase. In some embodiments, identifying the one or more interactions of the test compound, or the portion thereof, and the target condensate, or the component thereof, is based on comparing the partition characteristic of the test compound, or the portion thereof, for the target condensate; and the phase boundary characteristic of the component of the target condensate in the presence of the test compound, or the portion thereof. In some embodiments, identifying the one or more interactions of the test compound, or the portion thereof, and the target condensate, or the component thereof, is based on comparing the binding affinity characteristic of the test compound, or the portion thereof, for the component of the target condensate in the light phase; and the phase boundary characteristic of the component of the target condensate in the presence of the test compound, or the portion thereof. In some embodiments, identifying the one or more interactions of the test compound, or the portion thereof, and the target condensate, or the component thereof, is based on comparing the partition characteristic of the test compound, or the portion thereof, for the target condensate; the binding affinity characteristic of the test compound, or the portion thereof, for the component of the target condensate in the light phase; and the phase boundary characteristic of the component of the target condensate in the presence of the test compound, or the portion thereof.

In some embodiments according to any one of the methods described above, the interaction of the test compound, or the portion thereof, and the target condensate, or the component thereof, is selected from the group consisting of: (1) a preferential association of the test compound, or the portion thereof, and the component of the target condensate in the light phase as compared to a dense phase; (2) a preferential association of the test compound, or the portion thereof, and the component of the target condensate in the dense phase as compared to the light phase; (3) a preferential solubility of the test compound, or the portion thereof, in the dense phase of the target condensate as compared to the light phase; (4) a preferential solubility of the test compound, or the portion thereof, in the light phase as compared to the dense phase; (5) a preferential association of the test compound, or the portion thereof, and a feature in the dense phase of the target condensate as compared to the light phase; (6) an ability of the test compound, or the portion thereof, to compete with a phase-separation driving interaction for the component of the target condensate; (7) an ability of the test compound, or the portion thereof, to provide a phase-separation driving interaction for the component of the target condensate; (8) a preferential association of the test compound, or the portion thereof, and another component of the target condensate as compared to the component of the target condensate, wherein the preferential association of the test compound, or the portion thereof, and the other component of the target condensate hinders a phase-separation driving interaction for the component of the target condensate; (9) a preferential association of the test compound, or the portion thereof, and another component of the target condensate as compared to the component of the target condensate, wherein the preferential association of the test compound, or the portion thereof, and the other component of the target condensate provides a phase-separation driving interaction for the component of the target condensate; (10) a preferential association of the test compound, or the portion thereof, at a site of the component not involved in a phase-separation driving interaction as compared to a site of the component involved in a phase-separation driving interaction; and (11) a substantially equal association of the test compound, or the portion thereof, and the component of the condensate in both the light phase and the dense phase.

In some embodiments according to any one of the methods described above, the partition characteristic of the test compound, or the portion thereof, for the target condensate indicates the presence or absence of partitioning of the test compound, or the portion thereof, in the target condensate. In some embodiments, the presence or absence of partitioning of the test compound, or the portion thereof, in the target condensate is determined based on a partition characteristic threshold value. In some embodiments, the presence of partitioning of the test compound, or the portion thereof, in the target condensate is determined based on having the partition characteristic of more than 1.

In some embodiments according to any one of the methods described above, the partition characteristic of the test compound, or the portion thereof, for the target condensate indicates the degree of partitioning of the test compound, or the portion thereof, in the target condensate.

In some embodiments according to any one of the methods described above, the partition characteristic of the test compound, or the portion thereof, for the target condensate is based on a ratio of the test compound, or the portion thereof, in the dense phase of the target condensate versus the test compound, or the portion thereof, in the light phase.

In some embodiments according to any one of the methods described above, the binding affinity characteristic of the test compound, or the portion thereof, for the component of the target condensate in the light phase indicates the presence or absence of a binding association of the test compound, or the portion thereof, and the component of the target condensate in the light phase. In some embodiments, the presence or absence of the binding association is determined based on a binding affinity threshold value. In some embodiments, the presence of the binding association of the test compound, or the portion thereof, and the component of the target condensate in the light phase is determined based on having the binding affinity (e.g., K_{d}) of about 10 mM or less.

In some embodiments according to any one of the methods described above, the binding affinity characteristic of the test compound, or the portion thereof, for the component of the target condensate in the light phase indicates the degree of the binding association of the test compound, or the portion thereof, and the component of the target condensate in the light phase.

In some embodiments according to any one of the methods described above, the binding affinity characteristic of the test compound, or the portion thereof, for the component of the target condensate in the light phase is based on a dissociation constant (K_{d}) of the test compound, or the portion thereof, for the component of the target condensate in the light phase.

In some embodiments according to any one of the methods described above, the phase boundary characteristic of the component of the target condensate indicates the presence or absence of modulated partitioning of the component of the target condensate for the target condensate due to the presence of the test compound, or the portion thereof.

In some embodiments according to any one of the methods described above, the phase boundary characteristic is based on a phase diagram.

In some embodiments according to any one of the methods described above, identifying the one or more interactions of the test compound, or the portion thereof, and the target condensate, or the component thereof, based on comparing two or more of (i) the partition characteristic of the test compound, or the portion thereof, for the target condensate, (ii) the binding affinity characteristic of the test compound, or the portion thereof, for the component of the target condensate in the light phase, and (iii) the phase boundary characteristic of the component of the target condensate in the presence of the test compound, or the portion thereof, further comprises comparing to a reference. In some embodiments, the reference comprises information obtained using a reference compound regarding one or more of a partition characteristic of the reference compound for the target condensate, a binding affinity characteristic of the reference compound for the component of the target condensate in the light phase, and a phase boundary characteristic of the component of the target condensate in the presence of the reference compound. In some embodiments, the reference comprises information obtained using a plurality of (e.g., 2, 3, 4, 5, or more) reference compounds. In some embodiments, the plurality of reference compounds comprises compounds in the same chemical class as the test compound. In some embodiments, the plurality of reference compounds comprises compounds in different chemical classes as the test compound. In some embodiments, the plurality of reference compounds comprises at least 5 reference compounds.

In some embodiments according to any one of the methods described above, further comprising obtaining a mode of binding for the test compound and the component of the target condensate. In some embodiments, the mode of binding is determined via a polyphasic linkage formalism technique.

In some embodiments according to any one of the methods described above, further comprising measuring the partition characteristic of the test compound, or the portion thereof, for the target condensate. In some embodiments, measuring the partition characteristic of the test compound, or the portion thereof, for the target condensate comprises measuring the amount of the test compound, or the portion thereof, in the target condensate. In some embodiments, measuring the amount of the test compound, or the portion thereof, in the target condensate is determined via measuring the amount of the test compound, or the portion thereof, in an extra-condensate solution. In some embodiments, the partition characteristic of the test compound, or the portion thereof, for the target condensate is measured using a confocal microscopy or fluorescence spectroscopy technique. In some embodiments, the partition characteristic of the test compound, or the portion thereof, for the target condensate is measured by: (a) combining the test compound and a composition comprising or subjected to forming the target condensate and an extra-condensate solution; (b) obtaining a reference control; (c) measuring a mass spectrometry (MS) signal of the test compound in the extra-condensate solution, or a portion thereof, using an MS technique; (d) measuring an MS signal of the test compound in the reference control, or a portion thereof, using an MS technique; and (e) comparing the MS signal of the test compound from the extra-condensate solution and the MS signal of the test compound from the reference control.

In some embodiments according to any one of the methods described above, further comprising measuring the binding affinity characteristic of the test compound, or the portion thereof, for the component of the target condensate in the light phase. In some embodiments, measuring the binding affinity characteristic of the test compound, or the portion thereof, for the component of the condensate in the light phase comprises measuring the dissociation constant (K_{d}) of the test compound, or the portion thereof, for the component of the condensate in the light phase. In some embodiments, measuring the binding affinity characteristic of the test compound, or the portion thereof, for the component of the condensate in the light phase comprises using a MicroScale Thermophoresis (MST), isothermal titration calorimetry (ITC), surface plasmon resonance (SPR), nuclear magnetic resonance (NMR), or fluorescence polarization (FP) technique.

In some embodiments according to any one of the methods described above, further comprising measuring the phase boundary characteristic of the component of the target condensate due to the presence of the test compound, or the portion thereof. In some embodiments, the phase boundary characteristic of the component of the target condensate in the presence of the test compound, or the portion thereof, is measured using a microscopy, fluorescence spectroscopy, ultraviolet—visible (UV-Vis) spectroscopy, fluorescence recovery after photobleaching (FRAP), Static and Dynamic Light Scattering (SLS/DLS), or mass spectrometry-based technique.

In some embodiments according to any one of the methods described above, the phase boundary characteristic is representative of a partition characteristic of the component of the target condensate for the target condensate. In some embodiments, the phase boundary characteristic of the component of the target condensate in the presence of the test compound, or the portion thereof, is measured using a microscopy, fluorescence spectroscopy, ultraviolet—visible (UV-Vis) spectroscopy, fluorescence recovery after photobleaching (FRAP), Static and Dynamic Light Scattering (SLS/DLS), or mass spectrometry-based technique.

In some embodiments according to any one of the methods described above, the component of the target condensate is a macromolecule.

In some embodiments according to any one of the methods described above, the component of the target condensate comprises a polypeptide.

In some embodiments according to any one of the methods described above, the component of the target condensate comprises a nucleic acid.

In some embodiments according to any one of the methods described above, further comprising determining one or more contributing factors associated with a partition characteristic of the test compound, or the portion thereof, for a reference condensate. In some embodiments, the method further comprising comparing the one or more contributing factors associated with the partition characteristic of the test compound, or the portion thereof, for the target condensate with the one or more contributing factors associated with the partition characteristic of the test compound, or the portion thereof, for the reference condensate.

The present invention in another aspect provides a method of designing a compound having one or more desired interactions with a target condensate, or a component thereof, the method comprising: (a) identifying one or more interactions of a candidate compound, or a portion thereof, and the target condensate, or the component thereof, according to any one of the methods described above; and (b) designing the compound based on the candidate compound, or the portion thereof, associated with the identified one or more interactions.

The present invention in another aspect provides a method of designing a compound having a desired interaction profile, the method comprising modifying a precursor of the compound by attaching a moiety to the precursor, wherein the moiety comprises a characteristic having one or more desired interactions with a target condensate, or a component thereof, identified according to any one of the methods described above.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

All references cited herein, including patent applications and publications, are incorporated herein by reference in their entirety.

### BRIEF DESCRIPTION OF THE DRAWINGS

FIG. 1 shows an exemplary experimental strategy for identifying exemplary interactions of a test compound with a target condensate and/or a protein component thereof.

FIG. 2 show a theoretical plot of the partition characteristic of a compound for a condensate (PC-compound) versus phase boundary characteristic of a protein component represented by the partition characteristic of the protein component of the condensate for the condensate (PC-protein).

FIGS. 3A-3C show exemplary measurements from an analysis of Rhodamine B (RhoB) and a condensate comprising a FUS component using the methods described herein. FIG. 3A shows fluorescence spectroscopy (RhoB-FL) and mass spectroscopy (RhoB-MS) analysis of partitioning of two concentrations of RhoB for FUS-SNAP condensates formed in vitro, as reflected by the fraction of RhoB in the light phase extra-condensate solution (supernatant). FIG. 3B shows a binding affinity analysis of RhoB and FUS-SNAP in the light phase. FIG. 3C shows the modulation of phase behavior of FUS (FUS-EGFP) in the presence of RhoB by reducing partitioning of FUS (FUS-EGFP) into the condensate.

FIG. 4 shows a plot illustrating the partitioning of Rho800 and FUS (FUS-mEGFP) over a range of Rho800 concentrations, in FUS-containing condensates (FUS-mEGFP/RNA droplets).

FIG. 5A shows a three-dimensional model of FUS-RRM domain. FIG. 5B shows a plot indicating residues of FUS involved in binding of Rho800.

FIG. 6 shows a bar plot of partitioning of four compounds overlaid with information regarding binding association with condensate component. “NB” indicates “non-binder.”

### DETAILED DESCRIPTION

The present application provides, in some aspects, methods of identifying one or more interactions of a compound (or a portion thereof) and a condensate (or a component thereof). In some embodiments, the interaction is the manner in which the compound, or the portion thereof, and the condensate, or the component thereof, affect one another as evaluated in the dense phase and/or the light phase (also known as the dilute phase). In some embodiments, the interaction(s) include driving forces responsible, in whole or in part, for the partition characteristic of the compound, or the portion thereof, to the condensate—which may encompass various “causal factors” (such as contributing factors to the driving forces) such as: direct binding affinity between the compound, or the portion thereof, and the component of the condensate; preferential solubility for the compound, or a portion thereof, in the dense phase of the condensate (also known as the dense phase milieu or the dense phase microenvironment); and new binding sites existing predominantly in the dense phase. In some embodiments, the interaction(s) include driving forces responsible, in whole or in part, for a phase boundary characteristic, such as the partition characteristic, of the component of the condensate for the condensate in the presence of the compound, or the portion thereof—which may play a role in the resulting impact that the compound, or the portion thereof, has on a phase behavior of the condensate.

In certain aspects, the disclosure of the present application is based, at least in part, on the inventors' unique insights and findings regarding methods to identify the specific interactions (and more granular causal factors driving such interactions) between a compound, or a portion thereof, and a condensate, or components thereof. As described herein, individual measurements of: (i) a partitioning characteristic of a compound, or a portion thereof, for a condensate; (ii) a binding affinity characteristic of a compound, or a portion thereof, for a component of a condensate in the light phase; and (iii) a phase boundary characteristic of a component of a condensate in the presence of a compound, or a portion thereof, may provide convoluted and/or incomplete information regarding interactions between a compound, or a portion thereof, and a condensate, and/or a component thereof. Thus, using only such measurements in isolation (or without the taught methods described herein) may mask the one or more causal factors responsible for the interaction of a compound, or a portion thereof, and a condensate, or components thereof. For example, in some embodiments, the presence of partitioning of a compound, or a portion thereof, in a condensate is the result of a plurality of causal factors driving the interaction between the compound, or the portion thereof, and the condensate, or a component thereof, which results in the observed partitioning of the compound, or the portion thereof. Such causal factors can be, e.g., preferential binding of the compound (or portion thereof) to a binding motif of the condensate component exposed only in the dense phase, or a preference (such as higher solubility) of the compound (or portion thereof) for the dense phase environment, which can be a dense phase of the particular condensate, or a dense phase of any condensate, etc. Measuring partitioning alone does not identify such causal factors. Using the above measurements in isolation (or without the taught methods described herein) may also not reflect how a compound (or portion thereof) interacts with the condensate (or component thereof). For example, the presence of partitioning of a compound, or a portion thereof, in a condensate may modulate phase boundary characteristic of a component of the condensate, or does not affect the condensate (or component thereof) at all. As taught herein, comparison of any combination of (i) a partition characteristic of a compound, or a portion thereof, for a condensate; (ii) a binding affinity characteristic of the compound, or the portion thereof, for the component of the condensate in a light phase; or (iii) a phase boundary characteristic of the component of the condensate in the presence of the compound, or the portion thereof, enables the causal factor information to be deconvoluted, including partial deconvolution of properties of a compound. Thus, provided are methods for the identification of one or more specific interactions of the compound, or the portion thereof, and the condensate, or the component thereof. In some embodiments, the methods described herein can identify one or more driving forces involved in the interaction of a compound (or a portion thereof) and a condensate (or a component thereof). In some embodiments, the methods described herein can identify one or more driving forces that do not contribute to the interaction of a compound (or a portion thereof) and a condensate (or a component thereof).

In some instances, such methods allow for the identification of compounds, or portions thereof (such as via screening a compound library), having a desired interaction with a condensate, or a component thereof, such as having a desired partition characteristic. In some embodiments, such information can be used to guide further identification and/or design of one or more compounds. Thus, in some embodiments, provided is a method for identifying or designing a target compound with, e.g., improved potency, therapeutic index, and/or safety. Additionally, such methods enable rapid and accurate prediction of condensate-associated characteristics of a compound, or a portion thereof, based on an identified chemical motif or chemical class.

For purposes of exemplification, interactions between a compound and a condensate (or a component thereof), including partitioning of the compound into the condensate, can be influenced by, e.g., one or more of the following: (1) specific binding between the compound and a component of the condensate, wherein the conformation to which the compound binds is present in a component when in both a light phase (e.g., outside the condensate) and in a dense phase of the condensate (e.g., inside the condensate); (2) an increase in solubility of the compound in a dense phase of the condensate as compared to a light phase (i.e., this interaction is not driven by specific binding between the compound and a component of the condensate); (3) preferential binding of the compound and one or more targets that exist predominantly in a dense phase of the condensate (e.g., conformation of the component when in the dense phase of the condensate, newly formed binding features formed in the dense phase of the condensate, and/or presence of another molecule in the condensate); and (4) repulsive forces, such as a decrease in solubility of the compound in the light phase or a condition of the light phase that disfavors the compound, or a portion thereof. Interactions between a compound and a condensate may also play a role in the impact the compound has on the phase behavior of the condensate (or a component thereof), which includes changes in one or more of: dense phase composition (e.g., ratio of two or more components if present), phase separation (or disruption of phase separation) of a component of a condensate, saturation concentration of a component of a condensate, material properties (e.g., protein dynamics, viscosity), presence or absence of the condensate, and droplet morphology (e.g., sphericity, size, shape) of the condensate.

As taught herein, a method of identifying such interactions described herein enables further use of this information, including intelligent screening and/or design of compounds based on a desired compound activity, e.g., a desired partition characteristic and/or a desired impact the compound has on a condensate phase behavior. For example, it may be desirable to identify and/or design a compound that binds a specific target component of a condensate (e.g., a structured binding pocket, a linear motif, a transient structural feature), is preferentially partitioned into the condensate comprising the target component (i.e., is preferentially accumulated in the desired condensate), and that does not impact the phase behavior of the condensate (e.g., the compound will not impede the formation and/or presence of the condensate). The methods disclosed herein, which are useful for identifying such interactions, enable screening and/or design of compounds capable of any such desired activity, desired partition characteristic, and desired impact of the compound has on a condensate phase behavior.

In certain aspects, the disclosure of the present application is based, at least in part, on the inventors' unique insights and findings regarding methods of obtaining, such as measuring, a partition characteristic of a compound for a condensate (e.g., measuring the amount of a compound partitioned in a condensate), and uses thereof. In some aspects, the disclosure of the present application is based, at least in part, on the inventors' findings and developments regarding quantitative techniques, such as using mass spectrometry (MS), fluorescence spectroscopy, Raman spectroscopy, microscopy, and nuclear magnetic resonance (NMR), for determining a partition characteristic of a compound and/or a component of a condensate. Such methods allow for, e.g., accurate and reliable determination of a partition characteristic of a test compound for a target condensate in a rapid and high-throughput manner that is suitable for use in both simple and complex systems. Additionally, the described mass spectrometry-based methods are hypothesis-free (i.e., do not require a known, labeled compound or condensate, or a component thereof), compatible with a high-degree of compound multiplexing, do not require compound enrichment, can be used in homotypic and heterotypic systems, and can be performed with a low amount of compound and/or condensate components, which represents a more biologically relevant model and reduces the use of starting materials and reagents.

Thus, in some aspects, the present application provides a method of identifying one or more interactions of a test compound, or a portion thereof, and a target condensate, or a component thereof.

In other aspects, the present application provides a method of obtaining, such as determining or measuring, a partition characteristic of a compound, or a portion thereof, for a condensate; and a method of obtaining, such as determining or measuring, a phase boundary characteristic of a component of a condensate in the presence of a compound, or a portion thereof; and a method of obtaining, such as determining or measuring, a binding affinity characteristic of a compound, or a portion thereof, for the component of the condensate in a light phase.

In other aspects, the present application provides a library comprising a plurality of compounds (e.g., 2, 3, 4, or more compounds), wherein each compound of the plurality of compounds comprises a moiety associated with a desired interaction of the moiety and a target condensate and/or a component thereof, such as an interaction identified according to any of the methods described herein.

In other aspects, the present application provides a method of designing a compound having one or more desired interactions with a target condensate, or a component thereof, the method comprising: (a) identifying one or more interactions of a candidate compound, or a portion thereof, and the target condensate, or the component thereof, according to a method described herein, and (b) designing the compound based on the candidate compound, or the portion thereof associated with the identified one or more interactions.

In other aspects, the present application provides a method of designing a compound having a desired interaction profile, the method comprising modifying a precursor of the compound by attaching a moiety to the precursor, wherein the moiety comprises a characteristic having one or more desired interactions with a target condensate, or a component thereof, such as an interaction identified according to any of the methods described herein.

The section headings used herein are for organizational purposes only and are not to be construed as limiting the subject matter described. For example, some aspects of the disclosure are presented in a modular fashion, and such presentation is not to be construed as limited the possible combinations of approaches taught herein.

### I. Definitions

For purposes of interpreting this specification, the following definitions will apply and, whenever appropriate, terms used in the singular will also include the plural and vice versa. In the event that any definition set forth below conflicts with any document incorporated herein by reference, the definition set forth shall control.

As used herein, “condensate” means a non-membrane-encapsulated compartment formed by phase separation of one or more of proteins and/or other macromolecules (including all stages of phase separation).

The terms “polypeptide” and “protein,” as used herein, may be used interchangeably to refer to a polymer comprising amino acid residues, and are not limited to a minimum length. Such polymers may contain natural or non-natural amino acid residues, or combinations thereof, and include, but are not limited to, peptides, polypeptides, oligopeptides, dimers, trimers, and multimers of amino acid residues. Full-length polypeptides or proteins, and fragments thereof, are encompassed by this definition. The terms also include modified species thereof, e.g., post-translational modifications of one or more residues, for example, methylation, phosphorylation glycosylation, sialylation, or acetylation.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

### II. Methods of Identifying One or More Interactions of a Compound, or a Portion Thereof, and a Condensate, or a Component Thereof

In some aspects, provided herein is a method of identifying one or more interactions of a test compound, or a portion thereof, and a target condensate, or a component thereof. In some embodiments, the interaction is the manner in which the compound, or the portion thereof, and the condensate, or the component thereof, affect one another as evaluated in the dense phase and/or the light phase. In some embodiments, the interaction includes an aspect of the partition characteristic of the compound, or the portion thereof, for the condensate—which encompasses various causal factors associated with the condensate partitioning of the compound, or the portion thereof. In some embodiments, the interaction includes an aspect of the partition characteristic of the component of the condensate for the condensate in the presence of the compound, or the portion thereof—which encompasses various causal factors that play a role in the impact that the compound, or the portion thereof, has on the phase behavior of the condensate. In some embodiments, the compound is a test compound. In some embodiments, the compound is a reference compound (e.g., a compound known to modulate/not modulate the target condensate, or known to partition/not to partition into the target condensate, or known to bind/not to bind to a component of the target condensate). In some embodiments, the condensate is a target condensate. In some embodiments, the condensate is a reference condensate (e.g., a condensate known to be modulated/not modulated by the test compound, or known to have/not to have test compound partition).

In some embodiments, the method comprises obtaining, such as determining or measuring, a partition characteristic of a compound, or a portion thereof, for a condensate. In some embodiments, the partition characteristic of a compound, or a portion thereof, for a condensate is based on a ratio of the compound, or the portion thereof, in the dense phase of the condensate versus the compound, or the portion thereof, in a light phase (e.g., extra-condensate solution). In some embodiments, the compound is a test compound. In some embodiments, the compound is a reference compound. In some embodiments, the condensate is a target condensate. In some embodiments, the condensate is a reference condensate.

In some embodiments, the partition characteristic of the compound, or the portion thereof, for the condensate indicates (or is) the presence or absence of partitioning of the compound, or the portion thereof, into the condensate. In some embodiments, the partition characteristic of the compound, or the portion thereof, for the condensate indicates the degree (e.g., the amount) of partitioning of the compound, or the portion thereof, into the condensate. In some embodiments, the method comprises determining the presence or absence of partitioning of a compound, or a portion thereof, in a condensate. In some embodiments, the partition characteristic of more than 1 indicates that a compound, or a portion thereof, has a preference for the dense phase of a condensate (e.g., there are one or more attractive forces driving the compound, or the portion thereof, in the condensate; or there are one or more repulsive forces driving the compound, or the portion thereof, out of the light phase). In some embodiments, the partition characteristic of less than 1 indicates that a compound, or a portion thereof, has a preference for the light phase outside of a condensate (e.g., there are one or more repulsive forces driving the compound, or the portion thereof, out of the condensate; or there are one or more attractive forces driving the compound, or the portion thereof, in the light phase). In some embodiments, the partition characteristic of 1 indicates that a compound, or a portion thereof, does not have a preference for the light phase or the dense phase (e.g., the compound can freely diffuse through the condensate; there are no attractive or repulsive forces; attractive and repulsive forces counteract one another).

In some embodiments, the determination of the presence or absence of partitioning of a compound, or a portion thereof, in a condensate is based on a partition characteristic threshold value. One of skill in the art will appreciate that, in certain embodiments, a partition characteristic threshold value may depend on the technique used to obtain the partition characteristic, and can readily adjust the threshold value to correctly conclude whether a compound, or a portion thereof, partitions into a condensate, and to determine to what degree the compound, or the portion thereof, partitions into the condensate. In some embodiments, it may also be desirable to classify presence or absence based on satisfying a threshold value. For example, the threshold value may be based on the signal to noise ratio (S/N) associated with the technique used to determine the partition characteristic of the compound, or the portion thereof, for the condensate. In some embodiments, the threshold value enables the partition characteristic to be determined with a desired degree of confidence, e.g., a 5% or less false discovery rate (FDR).

In some embodiments, the presence of partitioning (e.g., preferential partitioning based on attractive forces driving the compound, or a portion thereof, into the condensate) is determined based on a compound partition characteristic of more than a partition characteristic threshold value (e.g., 1). In some embodiments, the partition characteristic is more than 1, such as about any of 2 or more, 3 or more, 4 or more, 5 or more, 10 or more, 15 or more, 20 or more, 25 or more, 30 or more, 35 or more, 40 or more, 45 or more, 50 or more, 60 or more, 70 or more, 80 or more, 90 or more, 100 or more, 200 or more, 300 or more, 400 or more, 500 or more, 600 or more, 700 or more, 800 or more, 900 or more, or 1,000 or more. In some embodiments, the absence of partitioning is determined based on a compound partition characteristic of less than a partition characteristic threshold value, such as a partition characteristic of less than 1. For example, in some embodiments, the partition characteristic of a compound for a condensate is measured using a fluorescence-based technique and the partition characteristic threshold value is about 2 (a measured partition characteristic of 2 or more indicates enrichment of the compound, or the portion thereof, in the condensate compared to in the light phase; a measured partition characteristic of less than 2 indicates non-enrichment of the compound, or the portion thereof, in the condensate compared to in the light phase). In some embodiments, the partition characteristic of a compound for a condensate is measured using a mass spectrometry-based technique (such as a mass spectrometry depletion assay) and the partition characteristic threshold value is about 2 (a measured partition characteristic of 2 or more indicates enrichment of the compound, or the portion thereof, in the condensate compared to in the light phase; a measured partition characteristic of less than 2 indicates non-enrichment of the compound, or the portion thereof, in the condensate compared to in the light phase). In some embodiments, the partition characteristic of a compound for a condensate is measured using a mass spectrometry-based technique (such as a mass spectrometry depletion assay) and the partition characteristic threshold value is about 50 (a measured partition characteristic of 50 or more indicates enrichment of the compound, or the portion thereof, in the condensate compared to in the light phase; a measured partition characteristic of less than 50 indicates non-enrichment of the compound, or the portion thereof, in the condensate compared to in the light phase). In some embodiments, the method comprises using a partition characteristic threshold value for determining the presence of partitioning, and using a second partition characteristic threshold value for determining the absence of partitioning.

In some embodiments, the presence (or lack thereof) of a compound, or a portion thereof, partitioning into a condensate is determined based on the degree of partitioning of the compound, or the portion thereof, into the condensate. For example, the degree of partitioning can be evaluated based on categories, such as by thresholds characterizing weak, medium and strong partitioning of a compound (a weak partitioner, a medium partitioner, and a strong partitioner, respectively). In some embodiments, the compound is characterized as a weak partitioner if the partition characteristic is less than 10. In some embodiments, the compound is characterized as a medium partitioner if the partition characteristic is equal to or more than 10 and less than 100. In some embodiments, the compound is characterized as a strong partitioner if the partition characteristic is 100 or more, such as 1000 or more.

In some embodiments, the presence of partitioning of a compound, or a portion thereof, in a condensate indicates any one or more of: (i) the compound, or the portion thereof, preferentially solvates in the dense phase of the condensate as compared to a light phase; (ii) the compound, or the portion thereof, associates, such as specifically binds, with the component of the condensate in the dense phase with a substantially similar affinity as compared to the component of the condensate in a light phase; (iii) the compound, or the portion thereof, preferentially associates, such as specifically binds, with the component of the condensate in the dense phase as compared to the component of the condensate in a light phase; and (iv) the compound, or the portion thereof, preferentially associates, such as specifically binds, with a feature in the dense phase, such as a conformation of the component of the condensate that is preferentially found in the condensate as compared to the light phase, or a new binding feature that preferentially exists in the condensate, such as presence of another molecule in the condensate or a site formed based on the association of two or more components of the condensate.

In some embodiments, the absence of partitioning of a compound, or a portion thereof, in a condensate (including absence based on not satisfying a threshold) indicates any one or more of: (i) the compound, or the portion thereof, does not solvate preferentially in the dense phase of the condensate as compared to a light phase; (ii) the compound, or the portion thereof, does not associate, such as specifically bind, to the component of the condensate, (iii) the compound, or the portion thereof, preferentially associates, such as specifically binds, with the component of the condensate that is preferentially found in the light phase as compared to the dense phase; and (iv) the compound, or the portion thereof, preferentially solvates in a light phase as compared to a dense phase of the condensate.

In some embodiments, the method comprises obtaining, such as determining or measuring, a binding affinity characteristic of a compound, or a portion thereof, for a component of a condensate in a light phase. In some embodiments, the compound is a test compound. In some embodiments, the compound is a reference compound. In some embodiments, the condensate is a target condensate. In some embodiments, the condensate is a reference condensate.

In some embodiments, the binding affinity characteristic of the compound, or the portion thereof, for the component of the condensate in the light phase indicates the presence or absence of a binding association (such as a specific binding affinity as measured via a dissociation constant) of the compound, or the portion thereof, and the component of the condensate in the light phase. In some embodiments, the binding affinity characteristic of the compound, or the portion thereof, for the component of the condensate indicates the degree of the binding association of the compound, or the portion thereof, and the component of the condensate in the light phase. In some embodiments, the binding affinity characteristic of the compound, or the portion thereof, for the component of the condensate in the light phase is based on a dissociation constant value (K_{d}) of the compound, or the portion thereof, for the component of the condensate in the light phase. In some embodiments, the method comprises determining the presence or absence of a binding association of a compound, or a portion thereof, for a component of a condensate in a light phase. In some embodiments, the determination of the presence or absence of binding is based on a binding affinity threshold value. One of skill in the art will appreciate that a binding affinity threshold value may depend on the technique used to obtain the binding affinity. In some embodiments, it may also be desirable to classify presence or absence of a binding association based on satisfying a threshold value. In some embodiments, the threshold value can readily be adjusted to conclude whether a compound, or a portion thereof, associates with a component of a condensate in the light phase, and/or to determine to what degree the compound, or the portion thereof, associates with the component of the condensate in the light phase. In some embodiments, the threshold value enables a binding affinity characteristic of the compound, or the portion thereof, with the component of the condensate in the light phase to be determined with a desired degree of confidence, e.g., a 5% or less FDR. In some embodiments, the threshold value is determined based on a minimum level of sensitivity of the technique used to assess the binding affinity.

In some embodiments, the presence of a binding association of the compound, or the portion thereof, and the component of the condensate in the light phase is determined based on a binding affinity (e.g., dissociation constant (K_{d})) of less than a binding affinity threshold value (e.g., 10 mM). In some embodiments, the binding affinity of the compound, or the portion thereof, and the component of the condensate in the light phase is about 10 mM or less, such as about any of 1 mM or less, 100 μM or less, 10 μM or less, 1 μM or less, 100 nM or less, 10 nM or less, 1 nM or less, 100 pM or less, 10 pM or less, or 1 pM or less.

In some embodiments, the presence (or lack thereof) of a binding association of the compound, or the portion thereof, and the component of the condensate in the light phase is determined based on the degree of binding affinity of the compound, or the portion thereof, and the component of the condensate in the light phase. For example, the degree of binding association can be evaluated based on categories, such as by thresholds characterizing weak, medium and strong binding of a compound (a weak binder, a medium binder, and a strong binder, respectively). In some embodiments, the compound is characterized as a weak binder if the K_{d} is equal to or greater than 100 μM. In some embodiments, the compound is characterized as a medium binder if the K_{d} is greater than or equal to 10 μM and less than 100 μM. In some embodiments, the compound is characterized as a strong binder if the Kd is less than 10 μM.

In some embodiments, the presence of a binding association of a compound, or a portion thereof, and a component of a condensate in the light phase indicates any one or more of: (i) the compound, or a portion thereof, associates, such as specifically binds, with the component of the condensate in the light phase, such as via a conformation of the component of the condensate preferentially present in the light phase; and (ii) the compound, or a portion thereof, associates, such as specifically or non-specifically binds, with the component of the condensate in the light phase and the dense phase. In some embodiments, the binding affinity characteristic of the compound (or portion thereof) and the component of the condensate in the light phase is further compared to a binding affinity characteristic of the compound (or portion thereof) and another macromolecule (e.g., another component of the condensate in the light phase), in order to determine whether the binding association of the compound (or portion thereof) and the component of the condensate in the light phase is specific or non-specific.

In some embodiments, the absence of a binding association of a compound, or a portion thereof, and a component of a condensate in the light phase (including absence based on not satisfying a threshold) indicates any one or more of: (i) the compound, or a portion thereof, does not associate, such as does not specifically bind, with the component of the condensate in the light phase, such as via a conformation of the component of the condensate preferentially present in the light phase; (ii) the compound, or a portion thereof, associates, such as specifically binds, with the component of the condensate in the dense phase, such as via a conformation of the component of the condensate preferentially present in the dense phase; and (iii) the compound, or a portion thereof, does not associate, such as does not specifically bind, with the component of the condensate either in light phase or in dense phase.

In some embodiments, the phase boundary characteristic of the component of the condensate indicates the presence or absence of modulated partitioning of the component of the condensate for the condensate due to the presence of the compound, or the portion thereof. In some embodiments, the method comprises obtaining, such as determining or measuring, a phase boundary characteristic of a component of a condensate in the presence of a compound, or a portion thereof. In some embodiments, the method comprises obtaining, such as determining or measuring, a phase boundary characteristic of a component of a condensate in the absence of a compound, or a portion thereof. In some embodiments, the phase boundary characteristic includes one or more pieces of information obtained from one or more phase diagrams. In some embodiments, the phase boundary characteristic indicates a partition characteristic of the component of the condensate for the condensate. In some embodiments, the phase boundary characteristic includes a phase boundary shift, such as a difference in phase diagrams due to a changing condition, such as presence/absence of a compound, or a portion thereof, or an amount thereof. In some embodiments, the method comprises obtaining, such as determining or measuring, a phase boundary shift of a component of a condensate due to the presence (such as an amount) of a compound, or a portion thereof. In some embodiments, the method comprises obtaining, such as determining or measuring a dose-dependent modulation in the phase boundary characteristic (e.g., saturation concentration) of the component of the condensate in the presence of the compound, or the portion thereof. In some embodiments, the compound is a test compound. In some embodiments, the compound is a reference compound. In some embodiments, the condensate is a target condensate. In some embodiments, the condensate is a reference condensate.

In some embodiments, the phase boundary characteristic, e.g., a phase boundary shift, of the component of the condensate due to the presence of the compound, or the portion thereof plays a role in the presence or absence of a modified phase behavior. In some embodiments, the modified phase behavior due to the presence of a compound, or a portion thereof, increases or decreases the ratio of a component of the condensate in the light phases versus the component of the condensate in the dense phase, as compared to when the compound, or the portion thereof, is absent. In some embodiments, the method comprises determining the presence or absence of a phase boundary shift of a component of a condensate due to the presence (such as an amount) of a compound, or a portion thereof. In some embodiments, the presence or absence of a phase boundary shift of a component of a condensate due to the presence of a compound, or a portion thereof, is based on an observed change in the phase behavior of a condensate (or component thereof), such as changes in one or more of: dense phase composition (e.g., what components are in a condensate, and selective exclusion of a component of a condensate, ratio of two or more components if present), material properties of the condensate (e.g., fluidity or protein dynamics, viscosity), phase separation (or disruption of phase separation) of a component of a condensate, saturation concentration of the component to form the condensate, presence or absence of the condensate, condensate morphology (e.g., size, shape, sphericity). In some embodiments, the method comprises determining the presence or absence of a partition characteristic of a component of a condensate for the condensate in the presence (such as an amount) of a compound, or a portion thereof. In some embodiments, the phase boundary characteristic of the component of the condensate in the presence of a compound indicates that the compound does not cause a phase boundary shift. In some embodiments, the phase boundary characteristic of the component of the condensate in the presence of a compound indicates that the compound causes a phase boundary shift.

In some embodiments, the presence or absence of a phase boundary characteristic is determined based on a phase boundary characteristic threshold value. One of skill in the art will appreciate that, in certain embodiments, a phase boundary characteristic threshold value may depend on the technique used to obtain the phase boundary characteristic, and can readily adjust the threshold value to correctly conclude whether, e.g., a component of a condensate partitions into a condensate, and to determine to what degree the component partitions into the condensate. In some embodiments, it may also be desirable to classify presence or absence of a phase boundary characteristic, such as a phase boundary shift, based on satisfying a threshold value. For example, the threshold value may be based on the signal to noise ratio (S/N) associated with the technique used to determine the partition characteristic of the component of a compound. In some embodiments, the threshold value enables the partition characteristic to be determined with a desired degree of confidence, e.g., a 5% or less FDR.

In some embodiments, the presence (or lack thereof) of modulation of phase boundary characteristic of a component of a condensate (e.g., partitioning of a component of a condensate into the condensate) in the presence of the compound, or the portion thereof is determined based on the degree of modulation of partitioning of the component into the condensate in the presence of the compound, or the portion thereof, such as measured via an EC₅₀ or the like. EC₅₀ (half maximal effective concentration) is the compound concentration required to induce 50% of the maximal observed effect on the condensate, such as to evict 50% of the component from the condensate, or to disrupt 50% of the condensates. For example, the degree of modulation of partitioning of a component into a condensate can be evaluated based on categories, such as by thresholds characterizing weak, medium, and strong modulation of partitioning of the component into the condensate by a component (a weak modulator, a medium modulator, and a strong modulator, respectively). In some embodiments, the compound is characterized as a weak phase boundary characteristic modulator if the EC₅₀ is 100 μM or more. In some embodiments, the compound is characterized as a medium phase boundary characteristic modulator if the EC₅₀ is equal to or more than 10 μM and less than 100 μM. In some embodiments, the compound is characterized as a strong phase boundary characteristic modulator if the EC₅₀ is less than 10 μM.

In some embodiments, the presence of a phase boundary shift of a component of the condensate in the presence of a compound, or a portion thereof, indicates any one or more of: (i) the compound, or the portion thereof, associates, such as specifically binds, with the component of the condensate in the light phase or dense phase, which results in competition with phase-separation driving interactions; (ii) the compound, or the portion thereof, associates, such as specifically binds, with the component of the condensate in the light phase or dense phase, which provides, amplifies, or adds to phase-separation driving interactions; and (iii) the compound, or the portion thereof, associates, such as specifically binds, with another component of the condensate in the light phase or dense phase, which either hinders phase-separation driving interactions, or provides, amplifies, or adds to phase-separation driving interactions.

In some embodiments, the absence of a phase boundary shift of a component of the condensate in the presence of a compound, or a portion thereof (including absence based on not satisfying a threshold) indicates any one or more of: (i) the compound, or the portion thereof, does not associate, such as specifically bind, with the component of the condensate in the light phase or dense phase; (ii) the compound, or the portion thereof, associates, such as specifically binds, with the component of the condensate at a site not involved with a phase-separation driving interaction; and (iii) the compound, or the portion thereof, does not substantially change characteristics of dense phase or the light phase.

As exemplified herein, information regarding each of (i) a partitioning characteristic of a compound, or a portion thereof, for a condensate, (ii) a binding affinity characteristic of a compound, or a portion thereof, for a component of a condensate in a light phase, and (iii) a phase boundary characteristic of a component of a condensate in the presence of a compound, or a portion thereof, may provide, e.g., convoluted information such as one or more causal factors of the interaction of the compound, or the portion thereof, and the condensate (or component thereof). For example, in some embodiments, the presence of partitioning of a compound, or a portion thereof, in a condensate is the result of a plurality of causal factors driving the interaction between the compound, or the portion thereof, and the condensate, or a component thereof, which results in the observed partitioning of the compound, or the portion thereof. For instance, interactions between a compound, or a portion thereof, and a condensate, or a component thereof (whether in the light phase or dense phase), may be influenced by, e.g., one or more of the following: (1) specific binding between the compound, or a portion thereof, and the component of the condensate, wherein the conformation to which the compound, or the portion thereof, associates is present in the component of the condensate when in both a light phase (e.g., outside the condensate) and in a dense phase of the condensate (e.g., inside the condensate); (2) an increase in solubility of the compound in a dense phase of the condensate as compared to a light phase (i.e., this interaction is not driven by specific binding between the compound and a component of the condensate); (3) preferential binding of the compound and one or more targets that exist predominantly in a dense phase of the condensate (e.g., conformation of the component when in the dense phase of the condensate, newly formed binding features formed in the dense phase of the condensate, and/or presence of another molecule in the condensate); and (4) repulsive forces, such as a decrease in solubility of the compound in the light phase or a condition of the light phase that disfavors the compound, or a portion thereof. Interactions between a compound and a condensate may also play a role in the impact the compound has on the phase behavior of the condensate (or component thereof), which includes dense phase composition, phase separation (or disruption of phase separation) of a component of a condensate, saturation concentration of a component of a condensate, material properties, presence or absence of the condensate, and droplet morphology of the condensate.

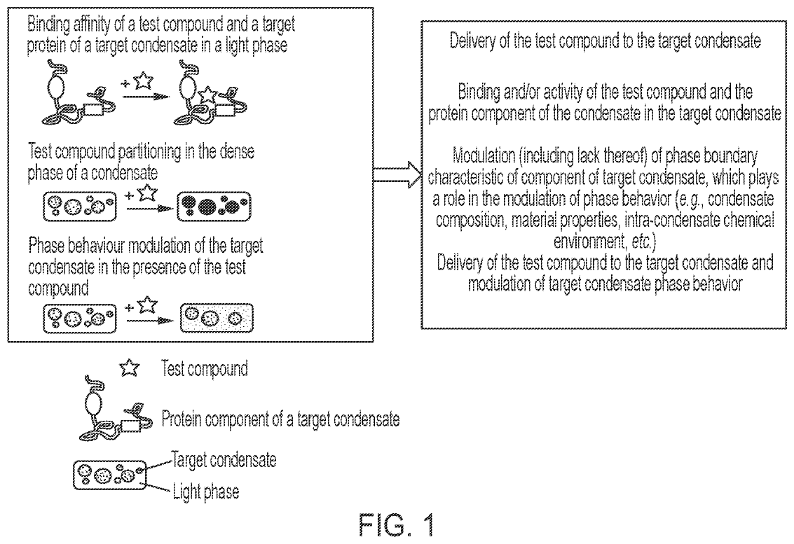

As taught herein, comparison of any combination of (i) a partition characteristic of a compound, or a portion thereof, for a condensate; (ii) a binding affinity characteristic of the compound, or the portion thereof, for the component of the condensate in a light phase; or (iii) a phase boundary characteristic of the component of the condensate in the presence of the compound, or the portion thereof, enables the interaction information (e.g., the causal factor(s) of the interaction) to be deconvoluted, thus allowing for the identification of one or more interactions of the compound, or the portion thereof, and the condensate, or the component thereof; also enables deconvolution (in full or in part) of properties of a compound. For example, as shown in FIG. 1, use of information from (i) a partition characteristic of a compound, or a portion thereof, for a condensate; (ii) a binding affinity characteristic of the compound, or the portion thereof, for the component of the condensate in a light phase; or (iii) a phase boundary characteristic of the component of the condensate in the presence of the compound, or the portion thereof (such as obtained via a phase behavior modulation), enables identification of one or more of, e.g., an aspect (e.g., moiety characteristic) of the compound that enables delivery of the compound to the target condensate, an aspect of the compound that enables binding of the compound and the component of the condensate in the condensate and/or activity of the compound, modulation (including lack thereof) of condensate phase behavior (e.g., condensate composition, material properties, intra-condensate chemical environment, etc.) based on a phase boundary characteristic of the component of the condensate, and delivery and/or activity of the compound to the condensate plus modulation of condensate phase behavior.

In some embodiments, identifying the one or more interactions of the compound, or the portion thereof, and the condensate, or the component thereof, is based on comparing a partition characteristic of the compound, or the portion thereof, for the condensate; and the binding affinity characteristic of the compound, or the portion thereof, for the component of the condensate in the light phase.

In some embodiments, identifying the one or more interactions of the compound, or the portion thereof, and the condensate, or the component thereof, is based on comparing the partition characteristic of the compound, or the portion thereof, for the condensate; and the phase boundary characteristic, such as the phase boundary shift, of the component of the condensate in the presence of the compound, or the portion thereof.

In some embodiments, identifying the one or more interactions of the compound, or the portion thereof, and the condensate, or the component thereof, is based on comparing the binding affinity characteristic of the compound, or the portion thereof, for the component of the condensate in the light phase; and the phase boundary characteristic, such as the phase boundary shift, of the component of the condensate in the presence of the compound, or the portion thereof.

In some embodiments, identifying the one or more interactions of the compound, or the portion thereof, and the condensate, or the component thereof, is based on comparing the partition characteristic of the compound, or the portion thereof, for the condensate; the binding affinity characteristic of the compound, or the portion thereof, for the component of the condensate in the light phase; and the phase boundary characteristic, such as the phase boundary shift, of the component of the condensate in the presence of the compound, or the portion thereof.

In some embodiments, the interaction of the compound, or the portion thereof, and the condensate, or the component thereof, is selected from one or more of: (i) a preferential association of the compound, or the portion thereof, and the component of the condensate in the light phase as compared to the dense phase; (ii) a preferential association of the compound, or the portion thereof, and the component of the condensate in the dense phase as compared to the light phase; (iii) a preferential solubility of the compound, or the portion thereof, in the dense phase of the condensate as compared to the light phase; (iv) a preferential solubility of the compound, or the portion thereof, in the light phase as compared to the dense phase; (v) a preferential association of the compound, or the portion thereof, and a feature in the dense phase of the condensate as compared to the light phase; (vi) an ability of the compound, or the portion thereof, to compete with a phase-separation driving interaction for the component of the condensate; (vii) an ability of the compound, or the portion thereof, to provide a phase-separation driving interaction for the component of the condensate; (viii) a preferential association of the compound, or the portion thereof, and another component of the condensate as compared to the component of the condensate, wherein the preferential association of the compound, or the portion thereof, and the other component of the condensate hinders a phase-separation driving interaction for the component of the condensate; (ix) a preferential association of the compound, or the portion thereof, and another component of the condensate as compared to the component of the condensate, wherein the preferential association of the compound, or the portion thereof, and the other component of the condensate provides a phase-separation driving interaction for the component of the condensate; (x) a preferential association of the compound, or the portion thereof, at a site of the component not involved in a phase-separation driving interaction as compared to a site of the component involved in a phase-separation driving interaction; and (xi) a substantially equal association of the compound, or the portion thereof, and the component of the condensate in both the light phase and dense phase. In some embodiments, the feature in the dense phase is another component of the condensate. In some embodiments, the feature in the dense phase is a new binding pocket formed in the condensate (or a binding pocket that is found predominantly in the dense phase as compared to the light phase), and includes new binding pockets within the component of the condensate and as formed by interactions of the component of the condensate with another component of the condensate. In some embodiments, the feature in the dense phase is a new configuration of the component of the condensate (or a configuration of the component that is found predominantly in the dense phase as compared to the light phase). In some embodiments, the feature in the dense phase is a favorable microenvironment formed in the condensate.

In some embodiments, the comparing of two or more of (i) a partition characteristic of a compound, or a portion thereof, for a condensate; (ii) a binding affinity characteristic of the compound, or the portion thereof, for the component of the condensate in a light phase; and (iii) a phase boundary characteristic of the component of the condensate in the presence of the compound, or the portion thereof, is performed according to the methods described herein based on the presence or absence of the partition characteristic of the compound, or the portion thereof, for the condensate; the presence or absence of the binding affinity characteristic of the compound, or the portion thereof, for the component of the condensate in the light phase; and/or the presence or absence of the phase boundary characteristic, such as the phase boundary shift, of the component of the condensate in the presence of the compound, or the portion thereof.

For example, in some embodiments, the presence of partitioning of the compound, or the portion thereof, into the condensate and the presence of binding association of the compound, or the portion thereof, for the component of the condensate in the light phase indicates that the partition characteristic of the compound (or portion thereof) for the condensate is based, at least in part, on the binding association of the compound (or portion thereof) for the component of the condensate. In some embodiments, the presence of a phase boundary characteristic, such as a phase boundary shift, of the component of the condensate due to the presence of the compound, or the portion thereof, further indicates that the compound, or the portion thereof, modulates the phase boundary of the component of the condensate (e.g., modulates the saturation concentration of the component for forming condensate). In some embodiments, the absence of a phase boundary characteristic, such as a phase boundary shift, of the component of the condensate due to the presence of the compound, or the portion thereof, further indicates that the compound, or the portion thereof, does not substantially modulate the phase boundary of the component of the condensate.

In some embodiments, the presence of partitioning of the compound, or the portion thereof, into the condensate and the absence of binding association of the compound, or the portion thereof, for the component of the condensate in the light phase indicates that the partition characteristic of the compound (or portion thereof) for the condensate is based, at least in part, on an increased solubility of the compound (or portion thereof) in the dense phase of the condensate as compared to the light phase and/or binding of the compound (or portion thereof) to a feature associated with the condensate (e.g., another component of the condensate). In some embodiments, the presence of a phase boundary characteristic, such as a phase boundary shift, of the component of the condensate due to the presence of the compound, or the portion thereof, further indicates that the compound, or the portion thereof, modulates the phase boundary of the component of the condensate. In some embodiments, the absence of a phase boundary characteristic, such as a phase boundary shift, of the component of the condensate due to the presence of the compound, or the portion thereof, further indicates that the compound, or the portion thereof, does not substantially modulate the phase boundary of the component of the condensate.

In some embodiments, the absence of partitioning of the compound, or the portion thereof, into the condensate and the presence of binding association of the compound, or the portion thereof, for the component of the condensate in the light phase indicates that the partition characteristic of the compound (or portion thereof) for the condensate is based, at least in part, on the decreased ability of the compound to bind the component of the condensate in the dense phase as compared to when the compound and the component of the condensate are in the light phase. In some embodiments, the presence of a phase boundary characteristic, such as a phase boundary shift, of the component of the condensate due to the presence of the compound, or the portion thereof, further indicates that the compound, or the portion thereof, modulates the phase boundary of the component of the condensate. In some embodiments, the absence of a phase boundary characteristic, such as a phase boundary shift, of the component of the condensate due to the presence of the compound, or the portion thereof, further indicates that the compound, or the portion thereof, does not substantially modulate the phase boundary of the component of the condensate.

In some embodiments, the absence of partitioning of the compound, or the portion thereof, into the condensate and the absence of binding association of the compound, or the portion thereof, for the component of the condensate in the light phase indicates that the partition characteristic of the compound (or portion thereof) for the condensate is based, at least in part, on no substantial increase in solubility of the compound in the condensate and/or no presence of substantial binding of the component of the condensate to a feature associated with the condensate. In some embodiments, the presence of a phase boundary characteristic, such as a phase boundary shift, of the component of the condensate due to the presence of the compound, or the portion thereof, further indicates that the compound, or the portion thereof, modulates the phase boundary of the component of the condensate. In some embodiments, the absence of a phase boundary characteristic, such as a phase boundary shift, of the component of the condensate due to the presence of the compound, or the portion thereof, further indicates that the compound, or the portion thereof, does not substantially modulate the phase boundary of the component of the condensate.

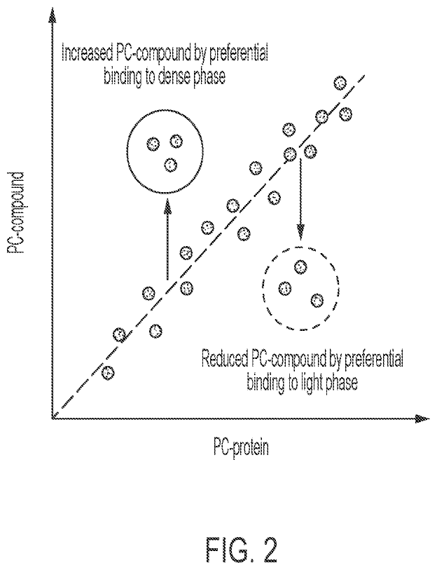

In some embodiments, the comparing of two or more of (i) a partition characteristic of a compound, or a portion thereof, for a condensate; (ii) a binding affinity characteristic of the compound, or the portion thereof, for the component of the condensate in a light phase; and (iii) a phase boundary characteristic of the component of the condensate in the presence of the compound, or the portion thereof, is performed according to the methods described herein based on the plotting coordinate values of two or more of: the partition characteristic of the compound, or the portion thereof, for the condensate; the binding affinity characteristic of the compound, or the portion thereof, for the component of the condensate in the light phase; and/or the phase boundary characteristic, such as a the phase boundary shift, of the component of the condensate in the presence of the compound, or the portion thereof. For example, as shown in FIG. 2, a plot of the partition characteristic of the compound, or the portion thereof, for the condensate (PC-compound) versus the phase boundary characteristic of the protein component of the condensate, as reflected by partitioning the protein component of the condensate in the presence of the compound, or the portion thereof (PC-protein), reveals a relationship that indicates one or more interactions of the compound, or the portion thereof, and the condensate, or the protein component thereof. In some embodiments, as shown in FIG. 2, compounds that bind to the protein component of a condensate in the light phase and dense phase without any preference will partition into the condensate at an amount proportional to the partitioning of the protein component into the condensate (see the exemplary dashed line representing this correlation). In some embodiments, increased PC-compound values indicate that the compound, or a portion thereof, preferentially binds to the protein component of the condensate in the dense phase as compared to the light phase. In some embodiments, decreased PC-compound values indicate that the compound, or a portion thereof, preferentially binds to the protein component of the condensate in the light phase as compared to the dense phase.

In some embodiments, the plot comprises the binding affinity characteristic (e.g., K_{d}) of the compound, or the portion thereof, for the component of the condensate in a light phase versus the partition characteristic of the compound, or the portion thereof, for the condensate. In some embodiments, coordinates on the plot for compounds (such as the test compound and/or reference compounds) whose partitioning into the dense phase of the condensate is driven only by binding to a pre-organized binding site and such binding is consistent in both the light phase and dense phases will create a derivable correlation (such as a linear or non-linear correlation). In some embodiments, the slope or shape of this correlation may differ across compounds from different chemical classes. In some embodiments, the test compound and the reference compounds belong to the same chemical class. In some embodiments, certain reference compounds belong to different chemical class as compared to the test compound. In some embodiments, within a chemical series, it is envisioned that compounds whose partitioning into the dense phase is affected by differential solubility, may be higher or lower on the plot than other analogs or derivatives in the chemical series and thus would fall above or below the observed correlation. In some embodiments, if some, but not all members of a chemical series, bind a discrete binding site with different intrinsic affinity in the light phase and dense phases, then these compounds may also be higher or lower on the plot than the correlation for that chemical series and cannot be distinguished from those that differ by virtue of different differential solubility in the light phase and dense phase. In some embodiments, when the partitioning of all members of a chemical series is affected to a similar extent, either by a difference in K_{d} for binding a discrete binding site on the component of the target condensate and/or differential solubility, all compounds would be expected to fall within the correlation on the plot, but the shape/slope/features of that curve may differ from curves for other chemical series, thereby allowing useful structural elements/moieties of those compounds that affect dense phase partitioning to be empirically discovered.

In some embodiments, the one or more interactions is determined based on comparing information regarding any combination of (i) a partitioning characteristic of a compound, or a portion thereof, for a condensate, (ii) a binding affinity characteristic of a compound, or a portion thereof, for a component of a condensate in the light phase, and (iii) a phase boundary characteristic of a component of a condensate in the presence of a compound, or a portion thereof, with a correlation derived from a reference. In some embodiments, the comparison comprises determining whether the information regarding the test compound is an outlier as compared to the correlation. In some embodiments, the correlation is derived from corresponding information of a plurality of reference compounds. In some embodiments, the method comprises determining an outlier based on an interquartile range (IQR) technique. In some embodiments, the method comprises determining an outlier based on a principal component analysis (PCA).

In some embodiments, identifying the one or more interactions of the compound, or the portion thereof, and the condensate, or the component thereof, based on the methods described herein comprises a comparison to a reference. In some embodiments, the reference comprises information obtained using a reference compound regarding one or more of a partition characteristic of the reference compound for the condensate, a binding affinity characteristic of the reference compound for the component of the condensate in the light phase, and a phase boundary characteristic of the component of the condensate in the presence of the reference compound. In some embodiments, the reference comprises information obtained using a plurality of reference compounds. In some embodiments, the plurality of reference compounds comprises compounds in the same chemical class as the test compound. In some embodiments, the plurality of reference compounds comprises compounds in different chemical classes as the test compound. In some embodiments, the plurality of reference compounds comprises at least about 5, such as at least about any of 10, 15, 20, 25, 30, 35, 40, 45, 50, 75, 100, 125, 150, 200, 250, 300, 400, 500, 100, 5,000, 10,000, 15,000, or 20,000, reference compounds.

In some embodiments, the method further comprises obtaining a mode of binding for the compound and the component of the condensate. In some embodiments, the mode of binding is derived from information regarding the partition characteristic of the compound, or the portion thereof, for the condensate, and the phase boundary characteristic (e.g., phase boundary shift) of the component of the condensate in the presence of the compound, or the portion thereof. In some embodiments, the mode of binding is derived from information regarding the binding affinity characteristic (e.g., K_{d}) of the compound, or the portion thereof, for a component of a condensate in the light phase, and the phase boundary characteristic (e.g., phase boundary shift) of the component of the condensate in the presence of the compound, or the portion thereof. In some embodiments, the mode of binding is based on direct binding via a sticker and/or linker, valency, or enhanced solubility. In some embodiments, the mode of binding is determined via a polyphasic linkage formalism technique.

### A. Obtaining a Partition Characteristic

In some aspects, provided herein are techniques for obtaining, such as determining or measuring, a partition characteristic of a compound, such as a test compound or a reference compound, for a condensate, such as a target condensate or a reference condensate. In some embodiments, the partition characteristic is a known value, such as obtained from previous experiments or a published literature value. In some embodiments, the partition characteristic is measured, such as using a method disclosed herein.

In some embodiments, obtaining, such as determining, the partition characteristic of a compound, or a portion thereof, for a condensate comprises measuring the amount of the compound, or the portion thereof, in the condensate. In some embodiments, measuring the amount of the compound, or the portion thereof, in the condensate is determined via determining, such as measuring, the amount of the compound, or the portion thereof, in an extra-condensate solution, such as a light phase.

In some embodiments, provided herein is a method for obtaining, such as determining or measuring, the partition characteristic of a compound, or a portion thereof, for a condensate. In some embodiments, the partition characteristic of the compound, or the portion thereof, for the condensate is based on a ratio of the compound, or the portion thereof, in the dense phase of the condensate versus the compound, or the portion thereof, in the light phase.

In some embodiments, measuring the partition characteristic of a compound, or a portion thereof, for a condensate comprises measuring the amount of the compound, or the portion thereof, in the condensate. In some embodiments, determining the amount of the compound, or the portion thereof, in the condensate determines the partition characteristic of the compound, or the portion thereof, for the condensate. In some embodiments, measuring the amount of the compound, or the portion thereof, in the condensate is determined via measuring the amount of the compound, or the portion thereof, in a system and outside of the condensate, such as in an extra-condensate solution. In some embodiments, the amount of the compound, or the portion thereof, in the system is known, and determining, such as measuring, the amount of the compound, or the portion thereof, outside of the condensate, such as in an extra-condensate solution, provides the amount of the compound partitioned in the condensate.

Techniques for determining the amount of a compound are known, and may utilized to facilitate the methods described herein. In some embodiments, determining the amount of the compound comprises quantifiably detecting the compound. In some embodiments, determining the amount of the compound comprises quantifiably detecting a compound label associated with the compound. In some embodiments, determining the amount of the compound comprises detecting an activity of the compound, thereby determining the amount of compound based on the detected activity. In some embodiments, the amount of compound is determined by mass spectrometry, liquid chromatography, and/or ultraviolet-visible spectrophotometry. In some embodiments, the amount of compound is determined by fluorescence microscopy. In some embodiments, standard curves may be used to aid in determining the amount of the compound. Alternatively or additionally, the amount of compound may be compared to a reference, such as a reference compound. In some embodiments, the reference compound is the compound label.

In some aspects, the methods described herein comprising determining an amount of a compound, such as a test compound or a reference compound, in a condensate are envisioned to encompass direct and indirect techniques for determining the amount of the compound in the condensate. In some embodiments, the amount of a compound in a condensate is determined directly. In some embodiments, the amount of a compound in a condensate is determined indirectly. In some embodiments, the amount of a compound in a condensate is determined via determining the amount of the compound not associated with the condensate, such as the amount of the compound in a light phase (such as an extra-condensate solution). In some embodiments, the amount of a compound in a condensate is determined via determining the amount of a reporter compound. In some embodiments, the reporter compound is associated with the condensate. In some embodiments, the reporter compound is not associated with the condensate.

In some embodiments, the amount of the compound, or the portion thereof, in the condensate can be compared to the amount of the compound, or the portion thereof, in other solutions or to the amount added to the system comprising the condensate and/or components thereof (e.g., a system provided with the same amount of compound and components but not subjected to condensate formation). Accordingly, in some embodiments, the method comprises comparing the amount of the compound, or the portion thereof, in the condensate to the amount added to the system; and/or the amount of the compound, or the portion thereof, in the extra-condensate solution; and/or the amount of the compound, or the portion thereof, in the cell; and/or the amount of the compound in another condensate. In some embodiments, the method comprises determining a ratio or percentage of the amount of the compound, or the portion thereof, depleted from the total system.

In some embodiments, the method comprises comparing the amount of the compound in the condensate to the amount of one or more components of the condensate. In some embodiments, comparing comprises determining a ratio or percentage of the amount of the compound in the condensate and the amount of one or more components of the condensate. In some embodiments, the method comprises determining the amount of the one or more components of the condensate in the condensate.

Techniques for forming condensates are known, and may be utilized to facilitate the methods described herein. For example, a condensate can be formed by altering the temperature of a composition comprising component(s) of the condensate, such as exposing the composition to lower or higher temperatures; by altering the salt content of the composition, such as diluting a salt in the composition or adding salt to the composition; by increasing the concentration of precursor macromolecules, such as adding a nucleic acid, e.g., RNA, in the composition; adding or changing a buffer in the composition; altering the ionic strength of the composition; altering the pH; adjusting the temperature; adding a stressor, such as arsenate; introducing or expressing a component of a condensate known to drive formation of the condensate; or adding a crowding agent, such as PEG or dextran. Some exemplary methods of forming condensates are also disclosed in Alberti et al., J Mol Biol, 430(23), 2018, 4806-4820, which is herein incorporated by reference.

In some embodiments, the method of determining the amount of the compound, or the portion thereof, comprises use of a mass spectrometry-based technique (e.g., to determine an amount of a compound in the light phase). In some embodiments, the method of determining the amount of the compound, or the portion thereof, comprises use of any one or more of a liquid chromatography (e.g., HPLC), microscopy (such a fluorescence microscopy or a confocal microscopy technique), quantitative image analysis, quantitative fluorescent microscopy and spectroscopy, nuclear magnetic resonance spectroscopy, Raman spectroscopy, and/or ultraviolet-visible spectrophotometry. One of ordinary skill in the art will readily appreciate that the system, and components thereof, may be selected to enable use of the technique for determining a partition characteristic of a compound, or a portion thereof, for a condensate. For example, when the technique comprises a fluorescence microscopy technique, the compound, or a portion thereof, and/or the condensate, or a component thereof, may be detected and quantified by the fluorescence microscopy technique.

In some embodiments, the method comprises admixing the compound, or the portion thereof, and a composition comprising the condensate, and/or the component thereof, and an extra-condensate solution. In some embodiments, the method comprises adding the compound, or the portion thereof, to a composition comprising the condensate, and/or the component thereof, and an extra-condensate solution. In some embodiments, the method comprises causing the formation of the condensate. In some embodiments, the method comprises admixing the compound, or the portion thereof, and a composition comprising the component of the condensate, and then subjecting the composition to a condensate-forming condition. In some embodiments, the method comprises admixing the compound, or the portion thereof, to a composition comprising component of the condensate, and then causing the formation of the condensate. In some embodiments, the composition comprising the component of the condensate, and/or the condensate, is subjected to a condensate-forming condition prior to being admixing with the compound, or the portion thereof. In some embodiments, the composition comprising the component of the condensate, and/or the condensate, is subjected to a condensate-forming condition after being admixing with the compound, or the portion thereof.

In some embodiments, the composition comprising the condensate, and/or the component thereof, comprises a cell. In some embodiments, the method comprises delivering the compound, or the portion thereof, to interior of the cell. In some embodiments, the method comprises causing the compound, or the portion thereof, to enter the cell.

In some embodiments, the method comprises separating the condensate from the extra-condensate solution, e.g., for the purpose of quantifying the compound, or the portion thereof, in the condensate and/or the extra-condensate solution. For example, in a cell-free composition, condensates may be sedimented or separated from the extra-condensate solution, e.g., using a centrifugation technique. Accordingly, in some embodiments, separating the condensate from the extra-condensate solution comprises separating the supernatant from the precipitate. In some embodiments, the method comprises centrifuging the composition. In some embodiments, the method comprises allowing the condensate to sediment.

In some embodiments, the method provided herein comprises adding two or more compounds to a composition comprising the condensate, and/or the component thereof, and an extra-condensate solution, for measuring the partition characteristic of one or more of the compounds, or a portion thereof, for a condensate. In some embodiments, the two or more test compounds are added sequentially or simultaneously.

In some embodiments, obtaining, such as determining, the partition characteristic of a compound, or a portion thereof, for a condensate comprises determining an amount of the compound, or the portion thereof, that is depleted from a system due to the presence and/or formation of the condensate. In some embodiments, the method comprises determining an amount of a compound, or a portion thereof, that is in an extra-condensate solution and not associated with a component of a condensate in the light phase. In some embodiments, the method comprises determining an amount of a compound, or a portion thereof, that is associated with, such as in, a condensate. In some embodiments, the method comprises determining an amount of a compound, or a portion thereof, that is associated with a component of a condensate in the light phase. In some embodiments, the amount of a compound, or a portion thereof, depleted from a system due to the presence and/or formation of a condensate is used to determine a condensate-associated characteristics, e.g., a partition characteristic of the compound for the condensate.

In some embodiments, the method comprises comparing a mass spectrometry (MS) signal of a compound, or a portion thereof, from an extra-condensate solution, or a portion thereof, and an MS signal of the compound, or the portion thereof, from a reference control, or a portion thereof, such as via a ratio of the MS signal of the compound, or the portion thereof, from the extra-condensate solution, or the portion thereof, and the MS signal of the compound, or the portion thereof, from the reference control, or the portion thereof. In some embodiments, the ratio of an MS signal of a compound, or a portion thereof, from an extra-condensate solution and an MS signal of the compound, or the portion thereof, from a reference control represents a depletion value. In some embodiments, the depletion value is representative of an amount of a compound, or a portion thereof, that is depleted from a system due to the presence and/or formation of a condensate. In some embodiments, the method further comprises obtaining, such as measuring an MS ion signal of a compound, or a portion thereof, in an extra-condensate solution, or a portion thereof, using a mass spectrometry technique. In some embodiments, the method further comprises obtaining, such as measuring an MS ion signal of a compound, or a portion thereof, in a reference control, or a portion thereof, using a mass spectrometry technique. In some embodiments, the method further comprises combining a compound, or a portion thereof, and a composition comprising a condensate and an extra-condensate solution. In some embodiments, the method further comprises causing the formation of a condensate in the presence of a compound, or a portion thereof, to obtain a composition comprising the condensate and an extra-condensate solution. In some embodiments, the method further comprises separating a condensate in a composition comprising the condensate and an extra-condensate solution, such as via pelleting the condensate in the composition or a particle separation technique. In some embodiments, the method further comprises obtaining, such as generating, a reference control.

In some embodiments, the amount of the compound, or the portion thereof, added to a composition comprising the condensate and an extra-condensate solution, or a precursor thereof such as a macromolecule, is based on an amount such that a relatively small depletion of the total amount of compound, or a portion thereof, from the extra-condensate solution can be determined (such as determined by comparing a measurement of the amount of the compound in an extra-condensate and a measurement of the amount of the compound in a reference control). In some embodiments, the amount of the compound, or the portion thereof, added to a composition comprising the condensate and an extra-condensate solution is based on the amount of the condensate, and/or one or more components thereof such as a macromolecule, in the composition. In some embodiments, the amount of the compound, or the portion thereof, added to a composition comprising the condensate and an extra-condensate solution is based on the compound capacity of the condensate. In some embodiments, the amount of the compound, or a portion thereof, is about 100 μM or less, such as about any of 90 μM, 80 μM, 70 μM. 60 μM, 50 μM, 40 μM, 30 μM, 20 μM, 10 μM, 9 μM, 8 μM, 7 μM, 6 μM, 5 μM, 4 μM, 3 μM, 2 μM, 1.5 μM, 1 μM, or less. In some embodiments, the amount of the compound, or a portion thereof, is about 1 μM or less, such as about any of 900 nM or less, 800 nM or less, 700 nM or less, 600 nM or less, 500 nM or less, 450 nM or less, 400 nM or less, 350 nM or less, 300 nM or less, 250 nM or less, 200 nM or less, 150 nM or less, 125 nM or less, 100 nM or less, 90 nM or less, 80 nM or less, 70 nM or less, 60 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, or 1 nM or less. In some embodiments, the lower limit of the compound, or the portion thereof, is based on the analytical method used to measure the amount of the compound, or the portion thereof. In some embodiments, the amount of macromolecule of the condensate in the composition is between about 1 nM and about 100 μM, such as about 1 μM and about 10 μM; and the compound, or a portion thereof, is about 100 μM or less, such as about any of 90 μM, 80 μM, 70 μM. 60 μM, 50 μM, 40 μM, 30 μM, 20 μM, 10 μM, 9 μM, 8 μM, 7 μM, 6 μM, 5 μM, 4 μM, 3 μM, 2 μM, 1.5 μM, 1 μM, or less, such as about any of 900 nM or less, 800 nM or less, 700 nM or less, 600 nM or less, 500 nM or less, 450 nM or less, 400 nM or less, 350 nM or less, 300 nM or less, 250 nM or less, 200 nM or less, 150 nM or less, 125 nM or less, 100 nM or less, 90 nM or less, 80 nM or less, 70 nM or less, 60 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, or 1 nM or less.

In some embodiments, the method comprises use of a reference control and/or methods of preparing the reference control. The reference controls described herein provide a reference measurement that is useful for determining an amount of a compound, or a portion thereof, that is depleted from a system due to the presence and/or formation of a condensate. In some embodiments, wherein a composition comprises: (a) an amount of a condensate, (b) an extra-condensate solution, (c) an amount, such as concentration, of a macromolecule of the condensate in the extra-condensate solution, and (d) an amount, such as a concentration, of a compound, or a portion thereof, in the composition, including (i) an amount of the compound, or the portion thereof, associated with, including in, the condensate, and (ii) an amount, such as a concentration, of the compound, or the portion thereof, in the extra-condensate solution, the reference control comprises the amount, such as concentration, of the macromolecule of the condensate in the extra-condensate solution, and the amount, such as a concentration, of the compound, or the portion thereof, in the composition. In some embodiments, the method comprises measuring the amount, such as concentration, of a macromolecule of a condensate in an extra-condensate solution, or a portion thereof, from a composition comprising the condensate and the extra-condensate solution.

In some embodiments, the reference control comprises an amount of a macromolecule of the condensate based on an amount of the macromolecule present in the extra-condensate solution after the composition has been subjected to pelleting of the condensate. In some embodiments, the reference control has the same concentration of the macromolecule as the concentration of the macromolecule in the extra-condensate solution after the composition has been subjected to pelleting of the condensate. In some embodiments, the reference control comprises the compound, or the portion thereof, at a concentration that is the same as that combined with the composition comprising the condensate and the extra-condensate solution. In some embodiments, the reference control is substantially free of a condensate. In some embodiments, the method further comprises determining the amount of the compound, or the portion thereof, in the reference control. In some embodiments, determining the amount of the compound, or the portion thereof, in the reference control comprises measuring the amount of the compound, or the portion thereof, in the reference control, or a portion thereof, using a mass spectrometry-based technique.

A variety of mass spectrometry-based techniques are suitable for the methods described herein. In some embodiments, the mass-spectrometry-based technique comprises measuring an MS signal of one or more ion species of one or more compounds. In some embodiments, the MS signal is of one or more ion species of a compound, or a portion thereof, such as one or more charge states of ions of the compound, or the portion thereof. In some embodiments, the MS signal is derived from a mass-to-charge (m/z) measurement. In some embodiments, the MS signal is ionization intensity. In some embodiments, the MS signal is peak height. In some embodiments, the MS signal is peak area, such as the integral of a signal corresponding with an MS ion signal. In some embodiments, the MS signal is peak volume, such as the integral of a signal corresponding with an MS ion signal. In some embodiments, the MS signal is a cumulative measurement of measured signals of ions of a compound, or a portion thereof. In some embodiments, the mass spectrometry-based technique comprises a liquid chromatography/mass spectrometry (LC/MS) technique, a liquid chromatography/tandem mass spectrometry (LC/MS) technique and/or a direct sample introduction technique (e.g., direct spray). In some embodiments, the mass spectrometry-based technique comprises gas chromatography/mass spectrometry (GC/MS). In some embodiments, the mass spectrometry-based technique comprises an acquisition technique selected from data-dependent acquisition, data-independent acquisition, selected reaction monitoring (SRM), and multiple reaction monitoring (MRM).

Liquid chromatography techniques contemplated by the present application include methods for separating macromolecules and/or compounds, or portion thereof, compatible with mass spectrometry techniques. In some embodiments, the liquid chromatography technique comprises a high performance liquid chromatography (HPLC) technique. In some embodiments, the liquid chromatography technique comprises a high-flow liquid chromatography technique. In some embodiments, the liquid chromatography technique comprises a low-flow liquid chromatography technique, such as a micro-flow liquid chromatography technique or a nano-flow liquid chromatography technique. In some embodiments, the liquid chromatography technique comprises an online liquid chromatography technique coupled to a mass spectrometer. In some embodiments, capillary electrophoresis (CE) techniques, or electrospray or MALDI techniques may be used to introduce the sample to a mass spectrometer. In some embodiments, direct sample introduction techniques may be used to introduce the sample to a mass spectrometer. In some embodiments, the mass spectrometry technique comprises an ionization technique. Ionization techniques contemplated by the present application include techniques capable of charging macromolecules and/or compounds, or portions thereof. In some embodiments, the ionization technique is electrospray ionization. In some embodiments, the ionization technique is nano-electrospray ionization. In some embodiments, the ionization technique is atmospheric pressure chemical ionization. In some embodiments, the ionization technique is atmospheric pressure photoionization. In some embodiments, the ionization technique is matrix-assisted laser desorption ionization (MALDI). In some embodiment, the mass spectrometry technique comprises electrospray ionization, nano-electrospray ionization, or a matrix-assisted laser desorption ionization (MALDI) technique.

Mass spectrometers contemplated by the present application, to which an online liquid chromatography technique may be coupled, include high-resolution mass spectrometers and low-resolution mass spectrometers. Thus, in some embodiments, the mass spectrometer is a time-of-flight (TOF) mass spectrometer. In some embodiments, the mass spectrometer is a quadrupole time-of-flight (Q-TOF) mass spectrometer. In some embodiments, the mass spectrometer is a quadrupole ion trap time-of-flight (QIT-TOF) mass spectrometer. In some embodiments, the mass spectrometer is an ion trap. In some embodiments, the mass spectrometer is a single quadrupole. In some embodiments, the mass spectrometer is a triple quadrupole (QQQ). In some embodiments, the mass spectrometer is an orbitrap. In some embodiments, the mass spectrometer is a quadrupole orbitrap. In some embodiments, the mass spectrometer is a Fourier transform ion cyclotron resonance (FT) mass spectrometer. In some embodiments, the mass spectrometer is a quadrupole Fourier transform ion cyclotron resonance (Q-FT) mass spectrometer. In some embodiments, the mass spectrometry technique comprises positive ion mode. In some embodiments, the mass spectrometry technique comprises negative ion mode. In some embodiments, the mass spectrometry technique comprises a time-of-flight (TOF) mass spectrometry technique. In some embodiments, the mass spectrometry technique comprises a quadrupole time-of-flight (Q-TOF) mass spectrometry technique. In some embodiments, the mass spectrometry technique comprises an ion mobility mass spectrometry technique. In some embodiments a low-resolution mass spectrometry technique, such as an ion trap, or single or triple-quadrupole approach is appropriate.

In some embodiments, the mass spectrometry-based technique comprises processing the obtained MS signals of the macromolecule and/or compounds, or portion thereof. In some embodiments, the mass spectrometry-based technique comprises peak detection. In some embodiments, the mass spectrometry-based technique comprises determining an ionization intensity. In some embodiments, the mass spectrometry-based technique comprises determining peak height. In some embodiments, the mass spectrometry-based technique comprises determining peak area. In some embodiments, the mass spectrometry-based technique comprises determining peak volume.

In some embodiments, the mass spectrometry-based technique comprises identifying the compound, or the portion thereof.

Thus, for example, in some embodiments, there is provided a method of determining a partition characteristic of a compound, or a portion thereof, in a condensate, the method comprising comparing an MS signal of ions of the compound, or the portion thereof, from an extra-condensate solution and an MS signal of ions of the compound, or the portion thereof, from a reference control, thereby determining the partition characteristic of the compound, or the portion thereof, in the condensate. In some embodiments, the method of determining a partition characteristic of the compound, or a portion thereof, in the condensate comprises: (a) obtaining, such as measuring, an MS signal of the compound, or the portion thereof, in the extra-condensate solution, or a portion thereof, using a mass spectrometry technique; (b) obtaining, such as measuring, an MS ion signal of the compound, or the portion thereof, in the reference control, or a portion thereof, using a mass spectrometry technique; and (c) comparing the MS signal of ions of the compound, or the portion thereof, from the extra-condensate solution and the MS signal of ions of the compound, or the portion thereof, from the reference control, thereby determining the partition characteristic of the compound, or the portion thereof, in the condensate.

In some embodiments, provided herein are methods of determining a condensate-associated characteristic of a small molecule compound, or a portion thereof, (such as a therapeutic small molecule that is 1,000 Da or less and/or satisfies Lipinski's rule of five) comprising determining an amount of the small molecule compound, or the portion thereof, that is depleted (including determining that there is a lack of depletion) from an extra-condensate solution due to the presence, formation, and/or modulation of a condensate. In some embodiments, there is provided a method of determining a partition characteristic of a small molecule compound, or a portion thereof, in a condensate, the method comprising comparing an MS signal of ions of the small molecule compound, or the portion thereof, from an extra-condensate solution and an MS signal of ions of the small molecule compound, or the portion thereof, from a reference control, thereby determining the partition characteristic of the small molecule compound, or the portion thereof, in the condensate. In some embodiments, the method of determining a partition characteristic of the small molecule compound, or the portion thereof, in the condensate comprises: (a) obtaining, such as measuring, an MS signal of the small molecule compound, or the portion thereof, in the extra-condensate solution, or a portion thereof, using a mass spectrometry technique; (b) obtaining, such as measuring, an MS ion signal of the small molecule compound, or the portion thereof, in the reference control, or a portion thereof, using a mass spectrometry technique; and (c) comparing the MS signal of ions of the small molecule compound, or the portion thereof, from the extra-condensate solution and the MS signal of ions of the small molecule compound, or the portion thereof, from the reference control, thereby determining the partition characteristic of the small molecule compound, or the portion thereof, in the condensate.

In some embodiments, provided herein are methods of determining a condensate-associated characteristic of a therapeutic compound (such as any of, or any combination of, an exogenous compound, a small molecule, a polypeptide, an oligonucleotide, a nucleic acid, an antibody, or fragment thereof, a synthetically produced compound, including cell culture produced compounds, or a compound that is not a condensate precursor macromolecule) comprising determining an amount of a therapeutic compound, or a portion thereof, that is depleted (including determining that there is a lack of depletion) from an extra-condensate solution due to the presence, formation, and/or modulation of a condensate. In some embodiments, there is provided a method of determining a partition characteristic of a therapeutic compound in a condensate, the method comprising comparing an MS signal of ions of the therapeutic compound, or the portion thereof, from an extra-condensate solution and an MS signal of ions of the therapeutic compound, or the portion thereof, from a reference control, thereby determining the partition characteristic of the therapeutic compound, or the portion thereof, in the condensate. In some embodiments, the method of determining a partition characteristic of the therapeutic compound, or the portion thereof, in the condensate comprises: (a) obtaining, such as measuring, an MS signal of the therapeutic compound, or the portion thereof, in the extra-condensate solution, or the portion thereof, using a mass spectrometry technique; (b) obtaining, such as measuring, an MS ion signal of the therapeutic compound, or the portion thereof, in the reference control, or the portion thereof, using a mass spectrometry technique; and (c) comparing the MS signal of ions of the therapeutic compound, or the portion thereof, from the extra-condensate solution and the MS signal of ions of the therapeutic compound, or the portion thereof, from the reference control, thereby determining the partition characteristic of the therapeutic compound, or the portion thereof, in the condensate.

In some embodiments, the method of determining a partition characteristic of a compound, or a portion thereof, in a condensate, the method comprising: (a) combining the compound, or the portion thereof, and a composition comprising or subjected to forming the condensate and an extra-condensate solution; (b) obtaining, such as preparing, a reference control; (c) measuring an MS signal of the compound, or a portion thereof, in the extra-condensate solution, or the portion thereof, using a mass spectrometry technique; (d) measuring an MS signal of the compound, or the portion thereof, in the reference control, or a portion thereof, using a mass spectrometry technique; (e) comparing the MS signal of the compound, or the portion thereof, from the extra-condensate solution and the MS signal of the compound, or the portion thereof, from the reference control, thereby determining the partition characteristic of the compound, or the portion thereof, in the condensate. In some embodiments, the method further comprises a step in between (a) and (c) of subjecting the composition to a condensate-formation condition to form the condensate and the extra-condensate solution in the presence of the compound.

In some embodiments, the method of determining a partition characteristic of a compound, or a portion thereof, in a condensate, the method comprising: (a) combining the compound, or the portion thereof, and a composition comprising or subjected to forming the condensate and an extra-condensate solution; (b) obtaining, such as preparing, a reference control; (c) measuring an MS signal of the compound, or the portion thereof, in the extra-condensate solution, or a portion thereof, using a mass spectrometry technique; (d) measuring an MS signal of the compound, or the portion thereof, in the reference control, or the portion thereof, using a mass spectrometry technique; (e) comparing the MS signal of the compound, or the portion thereof, from the extra-condensate solution and the MS signal of the compound, or the portion thereof, from the reference control, thereby determining the partition characteristic of the compound, or the portion thereof, in the condensate. In some embodiments, the method further comprises a step in between (a) and (c) of subjecting the composition to a condensate-formation condition to form the condensate and the extra-condensate solution in the presence of the compound.

In some embodiments, the method of determining a partition characteristic of a compound, or a portion thereof, in a condensate, the method comprising: (a) combining the compound, or the portion thereof, and a composition comprising or subjected to forming the condensate and an extra-condensate solution; (b) incubating the compound, or the portion thereof, and the composition (e.g., under a condensate-formation condition); (c) pelleting the condensate in the composition using a centrifugation technique; (d) obtaining, such as preparing, a reference control; (e) measuring an MS signal of the compound, or the portion thereof, in the extra-condensate solution, or a portion thereof, using a mass spectrometry technique; (f) measuring an MS signal of the compound, or the portion thereof, in the reference control, or the portion thereof, using a mass spectrometry technique; (g) comparing the MS signal of the compound, or the portion thereof, from the extra-condensate solution and the MS signal of the compound, or the portion thereof, from the reference control, thereby determining the partition characteristic of the compound, or the portion thereof, in the condensate.

In some embodiments, the method of determining a partition characteristic of a compound, or a portion thereof, in a condensate, the method comprising: (a) combining the compound, or the portion thereof, and a composition comprising or subjected to forming the condensate and an extra-condensate solution; (b) incubating the compound, or the portion thereof, and the composition (e.g., under a condensate-formation condition); (c) pelleting the condensate in the composition using a centrifugation technique; (d) obtaining, such as preparing, a reference control; (e) measuring an MS signal of the compound, or the portion thereof, in the extra-condensate solution, or a portion thereof, using a mass spectrometry technique; (f) measuring an MS signal of the compound, or the portion thereof, in the reference control, or a portion thereof, using a mass spectrometry technique; (g) comparing the MS signal of the compound, or the portion thereof, from the extra-condensate solution and the MS signal of the compound, or the portion thereof, from the reference control, thereby determining the partition characteristic of the compound, or the portion thereof, in the condensate.

In some embodiments of any of the methods or method steps described herein, the method is suitable for determining a condensate-associated characteristic for each of plurality of compounds, or a portion of each thereof, in a single composition comprising a condensate. For example, in some embodiments, there is provided a method comprising: (a) combining a plurality of compounds and a composition comprising the condensate and an extra-condensate solution; and (b) comparing an MS signal of ions of a first compound, or a portion thereof, of the plurality of compounds from an extra-condensate solution and an MS signal of ions of the first compound, or the portion thereof, from a reference control. In some embodiments, the MS signal of ions of each compound, or the portion thereof, of the plurality of compounds from an extra-condensate solution are compared with a respective MS signal of ions of each respective compounds, or the portion thereof, from a reference control. In some embodiments, the reference control comprises a plurality of compounds. In some embodiments, the number of compounds in the plurality of compounds is limited only by the capacity of the analytical method used for measuring the quantity of each compound. In some embodiments, the plurality of compounds comprises at least 5, such as at least any of 10, 15, 20, 25, 30, 35, 40, 45, 50, 75, 100, 125, 150, 175, 200, 250, 300, 350, 400, 450, 500, 600, 700, 800, 900, or 1,000, compounds. In some embodiments, the method further comprises obtaining, such as measuring, an MS signal of each of the compounds in the extra-condensate solution, or a portion thereof, using a mass spectrometry technique. In some embodiments, the method further comprises obtaining, such as measuring, an MS signal of each of the compounds in the reference control, or a portion thereof, using a mass spectrometry technique. In some embodiments, the MS signal of each of the compounds in either the extra-condensate solution or the reference control are obtained in a single mass spectrometry analysis.

In some embodiments, the techniques described herein may be applied to obtain, such as determine or measure, a partition characteristic of a component of a condensate for the condensate in the presence of a compound, such as a test compound or a reference compound.

### B. Obtaining a Binding Affinity Characteristic

In some aspects, provided herein are techniques for obtaining, such as determining or measuring, a binding affinity characteristic of a compound, such as a test compound or a reference compound, for a component of a condensate in a light phase (e.g., outside the condensate, in a non-condensed phase, extra-condensate solution). In some embodiments, the binding affinity characteristic is the direct binding association of a compound for a component of a condensate (e.g., the binding of a compound to a discrete binding site). In some embodiments, the binding affinity characteristic is the non-specific association of a compound for a component of a condensate. In some embodiments, the binding affinity characteristic is a dissociation constant (K_{d}). In some embodiments, the binding affinity characteristic is a known value, such as obtained from previous experiments or a published literature value. In some embodiments, the binding affinity characteristic is measured, such as using a method disclosed herein.

In some embodiments, the binding affinity characteristic is obtained, such as determined, by measuring the binding affinity characteristic (e.g., K_{d}) of the compound to the component of the condensate in an extra-condensate solution. In some embodiments, the binding affinity characteristic is obtained, such as determined, by measuring the binding affinity characteristic (e.g., K_{d}) of the compound to the component of the condensate without any condensate, or without providing any condensate formation condition (e.g., appropriate salt concentration, or stress).

In some embodiments, the binding affinity characteristic (such as binding affinity or binding association) is obtained, such as determined, by measuring the binding affinity characteristic of the compound, or a portion thereof, to the component of the condensate alone in the light phase. In some embodiments, the binding affinity characteristic is obtained, such as determined, by measuring the binding affinity characteristic of the compound, or a portion thereof, to the component of the condensate with the presence of one or more other components of the condensate in the light phase, such as one or more other components of the condensate known to co-exist in the condensate together with the test component of the condensate, or one or more other components of the condensate known to form a complex (e.g., the Mediator complex) with the test component of the condensate in vivo. In some embodiments, the one or more other components of the condensate are incubated with the test component of the condensate first (e.g., allow to form the complex similarly as that in vivo), then the compound (or the portion thereof) is provided, followed by measuring the binding affinity characteristic of the compound (or the portion thereof) to the test component of the condensate. In some embodiments, the test component of the condensate and the compound (or the portion thereof) are incubated first, then the one or more other components of the condensate are provided, followed by measuring the binding affinity of the compound (or the portion thereof) to the test component of the condensate.

“Binding affinity” generally refers to the strength of the sum total of binding interactions (e.g., non-covalent interactions) between a single binding site of a molecule (e.g., a chemical group of a compound) and its binding partner (e.g., a binding pocket of a component of the condensate). In some embodiments, unless indicated otherwise, “binding affinity” refers to intrinsic binding affinity that reflects a 1:1 interaction between members of a binding pair. Binding affinity can be indicated or reflected by K_{d}, K_{off}, Kₒₙ, or K_{d}. The term “K_{off}”, as used herein, is intended to refer to the off rate constant for dissociation of a compound from the compound/component of the condensate complex, as determined from a kinetic selection set up, expressed in units of s⁻¹. The term “Kₒₙ”, as used herein, is intended to refer to the on rate constant for association of a compound to the component of the condensate to form the compound/component of the condensate complex, expressed in units of M⁻¹s⁻¹. The term equilibrium dissociation constant “K_{D}” or “K_{d}”, as used herein, refers to the dissociation constant of a particular compound-component of the condensate interaction, and describes the concentration of compound required to occupy one half of all of the components of the condensate present in a solution of components of the condensate at equilibrium, and is equal to K_{off}/Kₒₙ, expressed in units of M. The measurement of K_{d} presupposes that all binding agents are in solution. In the case where the component of the condensate is immobilized, the corresponding equilibrium rate constant is expressed as half maximal effective concentration (EC₅₀), which gives a good approximation of K_{d}. The affinity constant, K_{d}, is the inverse of the dissociation constant, K_{d}, expressed in units of M⁻¹. The dissociation constant (K_{D} or K_{d}) is used as an indicator showing binding affinity of a compound to a component of the condensate. The K_{d} value that can be derived using these methods is expressed in units of M (mol/liter).

In some embodiments, obtaining, such as determining, the binding affinity characteristic of a compound, or a portion thereof, for a component of the condensate in the light phase comprises measuring dissociation constant (K_{d}) of the test compound, or the portion thereof, for the component of the condensate in the light phase. In some embodiments, the Kd of the binding between the compound, or the portion thereof, and the component of the condensate is about any of ≤10⁻¹M, ≤10⁻² M, ≤10⁻³ M, ≤10⁻⁴ M, ≤10⁻⁵ M, ≤10⁻⁶ M, ≤10⁻⁷ M, ≤10⁻⁸M, ≤10⁻⁹M, ≤10⁻¹⁰ M, ≤10⁻¹¹ M, or ≤10⁻¹² M.

The binding affinity characteristic of the compound, or the portion thereof, for the component of the condensate in the light phase can be measured by any appropriate method known in the art, such as MicroScale Thermophoresis (MST), isothermal titration calorimetry (ITC), surface plasmon resonance (SPR), nuclear magnetic resonance (NMR), fluorescence polarization (FP), or Fluorescence Resonance Energy Transfer (FRET) technique. Also see Vuignier et al. “Drug-protein binding: a critical review of analytical tools” (Anal Bioanal Chem, 2010) and Basturea, G. N. (“Biological Condensates,” MATER METHODS 2019; 9:2794) for exemplary methods.

For example, in an SPR assay (Biacore®), a component of the condensate can be coupled to the surface of a CM-5 sensor chip using EDC/NHS chemistry, then a compound (can be serial dilutions) is flown through and allowed to bind to the component of the condensate, and the response units (RU) bound by the compound is plotted against compound concentration to determine EC₅₀ values.

In some embodiments, the binding affinity characteristic of a compound, or a portion thereof, for a component of the condensate in the light phase is determined by Temperature Related Intensity Change (TRIC), such as by Protein Binding Affinity Analyzer—Dianthus. Briefly, the component of the condensate is labeled with a fluorescent dye and mixed with the compound, followed by an application of a very precise and brief laser-induced temperature change. If the compound binds to the component of the condensate, a variation in fluorescence intensity occurs and is amplified, which can be measured and plotted against compound concentration to obtain the dissociation constant or K_{d}.

In some embodiments, the binding affinity characteristic of a compound, or a portion thereof, for a target component of a condensate in the light phase is further compared to that of the compound, or the portion thereof, for one or more reference biomolecules in the light phase. In some embodiments, the one or more reference biomolecules co-exist with the target component in the condensate. In some embodiments, the one or more reference biomolecules do not form any condensate. In some embodiments, the one or more reference biomolecules form a different condensate that does not contain the target component. In some embodiments, the one or more reference biomolecules form a different condensate that also contains the target component. In some embodiments, the binding affinity characteristic of a test compound, or a portion thereof, for a component of a condensate in the light phase is further compared to that of a reference compound (e.g., one that has less or no affinity to the component in the light phase), or a portion thereof, for the component of the condensate.

### C. Obtaining a Phase Boundary Characteristic for a Condensate Component

In some aspects, provided herein are techniques for obtaining, such as determining or measuring, a phase boundary characteristic of a component of a condensate, such as a macromolecule, in the presence of a compound, such as a test compound or a reference compound. In some embodiments, the modulation of the phase boundary characteristic of the component of the condensate (e.g., phase boundary shift) due to the presence of the compound, or the portion thereof, plays a role in the presence or absence of a change in phase behavior of the condensate in the presence of the compound, or a portion thereof. In some embodiments, the presence or absence of a phase boundary shift of a condensate (e.g., a component of a condensate) due to the presence of a compound, or a portion thereof, is based on an observed change in the phase behavior of a condensate, such as changes in one or more of: dense phase composition (e.g., what component are in a condensate, selective exclusion of a component of a condensate, ratio of two or more components if present), phase separation (or disruption of phase separation) of a component of a condensate, saturation concentration of a component to form a condensate, physical properties of the condensate (e.g., fluidity or dynamics, viscosity, surface area), presence or absence of the condensate, condensate morphology (e.g., size, shape, sphericity). In some embodiments, the method comprises determining the presence or absence of a partition characteristic of a component of a condensate for the condensate in the presence (such as an amount) of a compound, or a portion thereof.

In some embodiments, the phase boundary characteristic is based on or represented in a phase diagram (e.g., saturation concentration, binodal curve, spinodal curve). In some embodiments, the phase diagram represents the concentration at which a component of a condensate undergoes phase separation under a phase separation control parameter, such as temperature, salt concentration, or concentration of a compound present. The phase transition line in the phase diagram is also referred to as phase boundary, indicating conditions (e.g., compound concentration, or condensate component concentration) under which dilute/light phase and dense phase can coexist at equilibrium. In some embodiments, a phase boundary shift can indicate a compound's modulation of the formation of a condensate. For purposes of illustration, in some embodiments, in the presence of a compound the saturation concentration of a condensate component becomes higher (i.e., more condensate components are now required to phase separate to form a condensate at the same phase separation control parameter), then the compound has inhibiting activity on condensate formation.

Techniques for obtaining a phase diagram are well known in the art, including but not limited to, fluorescence spectroscopy, turbidity/UV-Vis spectroscopy, fluorescence microscopy, dynamic light scattering, or static light scattering. In some embodiments, the phase boundary characteristic, such as the phase boundary shift of the component of the condensate due to the presence of the compound, or the portion thereof, is determined or measured in cells (e.g., by microscopy). In some embodiments, the phase boundary characteristic, such as the phase boundary shift of the component of the condensate due to the presence of the compound, or the portion thereof, is determined or measured in vitro.

In some embodiments, the compound (or a portion thereof) modulates the phase boundary characteristic of a target component of the condensate. In some embodiments, the compound (or a portion thereof) modulates the phase boundary characteristic of one or more other biomolecules. For example, in some embodiments, the compound (or a portion thereof) modulates the phase boundary characteristic of one or more other components of the condensate without changing that of the target component. In some embodiments, the compound (or a portion thereof) modulates the phase boundary characteristic of one or more other components of the condensate in additional to modulating that of the target component. In some embodiments, the compound (or a portion thereof) modulates the phase boundary characteristic of one or more other biomolecules, such that the one or more other biomolecules become additional components of the condensate (i.e., partition into the condensate), or compete with and/or replace the target component of the condensate. Thus in some embodiments, the method determines or measures the partition characteristic of the target component of a condensate for the condensate in the presence (such as an amount) of a compound, or a portion thereof. In some embodiments, the method determines or measures the partition characteristic of one or more other biomolecules (e.g., one or more other components of the condensate) for the condensate in the presence (such as an amount) of a compound, or a portion thereof.

In some embodiments, the presence of a phase boundary shift (e.g., as playing a role in a phase behavior change) indicates that the compound, or the portion thereof, is a phase modulator of the condensate. In some embodiments, the absence of a phase boundary shift indicates that the compound, or the portion thereof, is not a phase modulator of the condensate. In some embodiments, the presence or absence of a phase boundary shift is determined based on a phase shift threshold value of the compound (or portion thereof). In some embodiments, the presence or absence of a phase boundary shift is determined based on a phase shift threshold value of the condensate, such as the target component of the condensate, or one or more other components of the condensate.

The phase boundary characteristic, such as phase boundary shift of a component of the condensate due to the presence of a compound, or a portion thereof, can be determined or measured by methods known in the art, such as microscopy (e.g., to study partition amount of a component to the condensate), fluorescence spectroscopy (such as fluorescence correlation spectroscopy (FCS)), fluorescence recovery after photobleaching (FRAP, e.g., for studying fluidity changes of the condensate), ultraviolet-visible (UV-Vis) spectroscopy, small-angle x-ray scattering, or Static and Dynamic Light Scattering (SLS/DLS) technique. For example, light scattering methods like dynamic light scattering (DLS), static light scattering (STS) and small-angle light scattering (SLS) can be used to determine the size and shape of condensates. Component analysis such as immunofluorescence, fluorescence in situ hybridization (FISH), mass spectrometry (MS), RNA-seq, NMR spectroscopy, can also be applied to study phase behavior change of a condensate, particularly condensate composition changes or composition changes of the extra-condensate solution in the presence of a compound (or a portion thereof). Also see various in vitro and in cell condensate analysis methods described in Basturea, G. N. (“Biological Condensates,” MATER METHODS 2019; 9:2794).

In some embodiments, the phase boundary shift of the component of the condensate due to the presence of the compound, or the portion thereof, is determined or measured by compound dose-dependent phase boundary shift assay. In some embodiments, a component of the condensate and a compound (or a portion thereof) are incubated together, then condensate formation condition (e.g., appropriate salt concentration, or stress) is provided, and the effect of the compound (or a portion thereof) on phase boundary characteristic of the condensate (or the component of the condensate) is observed, such as measuring partition characteristic of the condensate component. In some embodiments, a component of the condensate and various concentrations of a compound (or a portion thereof) are incubated together, then a condensate formation condition (e.g., appropriate salt concentration, or stress) is provided, and the effect (e.g., dose-dependent effect) of the compound (or a portion thereof) on phase boundary characteristic of the component of the condensate is determined. In some embodiments, the compound concentration where the phase boundary characteristic change(s) initially occurs is recorded as a phase boundary shift threshold value (e.g., the amount of the compound, or the portion thereof, needed to modulate phase boundary characteristic). In some embodiments, various concentrations of a component of the condensate and a fixed concentration of a compound (or a portion thereof) are incubated together, then a condensate formation condition (e.g., appropriate salt concentration, or stress) is provided, and the effect of the compound (or a portion thereof) on phase boundary characteristic of the condensate (or the component of the condensate) is determined. In some embodiments, the condensate component concentration where the phase boundary characteristic change(s) initially occurs is recorded as a phase boundary shift threshold value. In some embodiments, the phase boundary shift threshold value is the EC₅₀ concentration.

As used herein, “EC₅₀,” is intended to refer to the concentration of a substance (e.g., a compound) that is required for 50% activation or enhancement (or inhibition) of a biological process, a biochemical process, or a biophysical process, or component of a process. For example, EC₅₀ can refer to the concentration of a compound that provokes (or inhibits) a response halfway between the baseline and maximum response in an appropriate assay of the target activity.

In some embodiments, the component of the condensate is allowed to form a condensate (e.g., under appropriate salt concentration, or stress), then the compound (or a portion thereof) is provided to the condensate, and the effect of the compound (or a portion thereof) on phase boundary characteristic of the condensate (or the component of the condensate) is observed, such as measuring partition characteristic of the condensate component. In some embodiments, a component of the condensate is allowed to form a condensate (e.g., under appropriate salt concentration, or stress), then various concentrations of a compound (or a portion thereof) are provided to the condensate (e.g., in separate systems), and the effect (e.g., dose-dependent effect) of the compound (or a portion thereof) on phase boundary characteristic of the condensate (or the component of the condensate) is determined. In some embodiments, various concentrations of a component of the condensate are allowed to form a condensate (e.g., under appropriate salt concentration, or stress, and in separate systems), then a fixed amount of a compound (or a portion thereof) is provided to the condensate, and the effect of the compound (or a portion thereof) on phase boundary characteristic of the condensate (or the component of the condensate) is determined.

In some embodiments, the partition characteristic of a component of a condensate (e.g., the target component, or one or more other biomolecules that become components of the condensate in the presence of the compound or portion thereof) is determined by measuring the amount of the component that is inside the condensate (intensity-in, “I_{component-in}”), and/or by measuring the amount of the component that is outside of the condensate (i.e., extra-condensate solution; intensity-out, “I_{component-out}”). In some embodiments, the partition characteristic of a component of a condensate is calculated as I_{component-in} divided by I_{component-out}, referred to as “PC_{component}.” The partition characteristic of a component of a condensate can also be calculated as I_{component-in} divided by I_{component-total}, I_{component-out} divided by I_{component-total}, or I_{component-out} divided by I_{component-in}.

In some embodiments, the phase boundary characteristic, such as a phase boundary shift, of a component of a condensate due to the presence of the compound, or the portion thereof, is obtained by determining or measuring one or more of the phase behavior characteristics, including but are not limited to: (i) number of condensates comprising and/or not comprising a component (e.g., target component, or one or more other biomolecules that become components of the condensate in the presence of the compound or portion thereof); (ii) size, shape, and/or sphericity of the condensates; (iii) location of the condensates (e.g., in cell assay); (iv) location of a component of a condensate (e.g., the target component is more attracted into the condensate or excluded from the condensate, such as relocating from the condensate to another organelle in the cell; or one or more other biomolecules become partitioned into the condensate in the presence of the compound or portion thereof); (v) surface area of the condensates; (vi) composition of the condensates; (vii) liquidity (or dynamic) of the condensates (e.g., measure by FRAP); (viii) solidification of the condensates; (ix) dissolution of the condensates; (x) presence and/or amount of fiber formation; (xi) partitioning of a condensate component into the condensates; and (xii) aggregation of a component of a condensate.

In some embodiments, the phase boundary characteristic, such as phase boundary shift, of a target component of a target condensate due to the presence of a test compound, or the portion thereof, is further compared to the phase boundary characteristic, such as phase boundary shift, induced by the presence of the reference compound, or the portion thereof. In some embodiments, the phase boundary characteristic, such as phase boundary shift, of a target component of a target condensate due to the presence of the test compound, or the portion thereof, is further compared to that of a component of a reference condensate due to the presence of the test compound, or the portion thereof. In some embodiments, the phase boundary characteristic, such as phase boundary shift, of a target component of a target condensate due to the presence of the test compound, or the portion thereof, is further compared to that of a reference component of the target condensate (e.g., another biomolecule that is not phase-shifted by the compound) due to the presence of the test compound, or the portion thereof.

### D. Obtaining a Mode of Binding

In some aspects, provided herein are techniques for obtaining, such as determining or measuring, a mode of binding for a compound, such as a test compound or a reference compound, and a condensate (e.g., a component of a condensate), such as a target condensate or a reference condensate. In some embodiments, the mode of binding is derived from information regarding the partition characteristic of the compound, or the portion thereof, for the condensate (e.g., a component of the condensate), and the phase boundary shift of the component of the condensate in the presence of the compound, or the portion thereof. In some embodiments, the mode of binding is derived from information regarding the binding affinity of the compound, or the portion thereof, for a component of a condensate in the light phase, and the phase boundary shift of the component of the condensate in the presence of the compound, or the portion thereof. In some embodiments, the mode of binding is derived from information regarding the partition characteristic of the compound, or the portion thereof, for the condensate (e.g., a component of the condensate), the binding affinity of the compound, or the portion thereof, for a component of the condensate in the light phase, and the phase boundary shift of the component of the condensate in the presence of the compound, or the portion thereof. In some embodiments, the mode of binding is based on direct binding via a sticker and/or linker, valency, or enhanced solubility. In some embodiments, the mode of binding is determined via a polyphasic linkage formalism technique. In some embodiments, the mode of binding is determined via PCA.

Any methods known in the art that can be used for mode of binding analysis, such as polyphasic linkage formalism, linkage analysis, PCA, multiscale simulations, or hierarchical clustering. Polyphasic linkage formalism can be applied similarly as described in Tisel et al. (“Polyphasic linkage between protein solubility and ligand binding in the hemoglobin-polyethylene glycol system,” J Biol Chem, 1980, 255(19):8975-8), Gill et al. (“Ligand-linked phase equilibria of sickle cell hemoglobin,” J Mol Biol. 1980, 140(2):299-312), Ruff et al. (“Ligand effects on phase separation of multivalent macromolecules,” Proc Natl Acad Sci USA. 2021 Mar. 9; 118(10):e2017184118), or Posey et al. (“Profilin reduces aggregation and phase separation of huntingtin N-terminal fragments by preferentially binding to soluble monomers and oligomers,” J Biol Chem, 2018, 293(10):3734-3746), which studies the relationship between binding affinity in the light phase and phase behavior. Also see the methods of establishing a numerical stickers-and-spacers model for studying the correlation of valence and amino acid sequence with protein phase behavior (Martin et al., “Valence and patterning of aromatic residues determine the phase behavior of prion-like domains,” Science 2020; 367(6478):694-699), and polyphasic interaction thermodynamic analysis (PITA) which can be broadly utilized to extract thermodynamic parameters from microscopy images (Riback et al., “Composition dependent phase separation underlies directional flux through the nucleolus,” bioRxiv, 2019; Riback et al., “Composition-dependent thermodynamics of intracellular phase separation,” Nature. 2020 May; 581(7807): 209-214).

### E. Assay Systems

Condensates can be formed and studied using the methods described herein in a variety of settings and assay systems. In some embodiments, the method comprises an in vivo assay. In some embodiments, the assay is a cell-based assay. In some embodiments, the method comprises an in vitro assay. In some embodiments, the in vitro assay comprises use of a human-generated condensate. In some embodiments, the method comprises use of both an in vivo and an in vitro assay.

In some embodiments, the assay system comprises a composition, wherein the composition comprises any one or more of the compound, or the portion thereof, the condensate, the extra-condensate solution, or the component of the condensate. In some embodiments, the composition comprises a cell. In some embodiments, the condensate, or the component thereof, is in the cell. In some embodiments, the extra-condensate solution is intracellular fluid, such as cytosol or nucleosol. In some embodiments, the condensate, or the component thereof, is not in the cell. In some embodiments, the condensate is an extra-cellular condensate, such as a condensate in the extra-cellular matrix. In some embodiments, the extra-cellular fluid is interstitial fluid or plasma. In some embodiments, the method comprises causing, such as triggering, the formation of a condensate.

In some embodiments, the cell is derived from or located in a microorganism or an animal cell. In some embodiments, cell is a human cell. In some embodiments, the cell is a neuron. In some embodiments, the cell is a cancer cell. In some embodiments, the cell is or is derived from induced pluripotent stem cells (iPS cells), HeLa cells, or HEK293 cells. In some embodiments, the cell comprises a condensate, or a component thereof, that is determined to be dysregulated compared to a normal healthy state. In some embodiments, the cell comprises a mutation associated with a disease, such as a genetic mutation. In some embodiments, the cell expresses a mutation associated with a disease, such as a mutated polypeptide. In some embodiments, the cell has one or more features of a neurodegenerative or proliferative disease. In some embodiments, the cell has been treated with arsenate (and/or another compound known to modulate a condensate), a temperature change, or a pH change. In some embodiments, the cell expresses a protein that is labeled with a fluorescent component, such as a fluorescent polypeptide. In some embodiments, the protein is a protein known to concentrate in the condensate.

In some embodiments, the composition does not comprise a cell. In some embodiments, the composition is a cell-free composition. In some embodiments, the composition comprises non-phase separated components of the condensate, which are capable of being incorporated into a condensate and/or have been incorporated into a condensate. In some embodiments, the composition comprises a buffer. In some embodiments, the composition comprises a component useful for formation of the condensate, such as one or more salts, and/or one or more crowding agents (e.g., PEG or dextran).

In some embodiments, the composition comprises a plurality of condensates. In some embodiments, the plurality of condensates comprises condensates of a single species of a condensate (e.g., as defined by shared types of components of the condensates or a common function). In some embodiments, the plurality of condensate comprises two or more species of a condensate.

### F. Compounds, Condensates, and Components of Condensates

The methods described herein are generally applicable to a diverse array of compounds (such as test compounds and reference compounds), condensates (such as target condensates and reference condensates), and components of condensates, such as macromolecules.

In some embodiments, the compound, such as a test compound or a reference compound, is a small molecule, a polypeptide, a peptidomimetic, a lipid, a nucleic acid, or any combination thereof. In some embodiments, the compound has a molecular weight of less than 1,000 Da, such as 500 Da or less. In some embodiments, the compound satisfies Lipinski's rule of five. In some embodiments, the compound is a small molecule (such as a therapeutic small molecule that is 1,000 Da or less and/or satisfies Lipinski's rule of five). In some embodiments, the compound comprises a nucleic acid. In some embodiments, the compound comprises RNA, such as a siRNA, miRNA, mRNA, or lnRNA, or an analog thereof. In some embodiments, the compound comprises DNA, or an analog thereof. In some embodiments, the compound is a non-naturally occurring compound. In some embodiments, the compound is an exogenous compound. In some embodiments, the compound comprises a polypeptide. In some embodiments, the compound comprises an antibody. In some embodiments, the compound is a therapeutic compound approved by a regulatory agency, such as an agent approved for medical treatment by the United States Food and Drug Administration (FDA). In some embodiments, the compound is a novel compound.

In some embodiments, the compound, or a portion thereof, is charged. In some embodiments, the compound, or a portion thereof, is hydrophobic. In some embodiments, the compound, or a portion thereof, is hydrophilic. In some embodiments, the compound, or a portion thereof, comprises an alkaloid, a glycoside, a phenazine, a phenol, a polyketide, a terpene, or a tetrapyrrole.

In some embodiments, the compound comprises a label. In some embodiments, the label is a radioactive label, a colorimetric label, a luminescent label, a chemically-reactive label (such as a component moiety used in click chemistry), or a fluorescent label. In some embodiments, the compound is a small molecule comprising a label. In some embodiments, the compound is a small molecule comprising a fluorophore. In some embodiments, the compound is a polypeptide comprising a label. In some embodiments, the compound is a polypeptide comprising a fluorophore. In some embodiments, the compound is a nucleic acid comprising a label. In some embodiments, the compound is a nucleic acid comprising a fluorophore. The compound label can be conjugated to the compound covalently or non-covalently.

In some embodiments, the condensate, such as a target condensate or reference condensate, is an in vivo condensate. In some embodiments, the condensate is a cellular condensate. In some embodiments, the condensate is an extra-cellular condensate. In some embodiments, the condensate is an in vitro condensate. In some embodiments, the condensate is a model for an in vivo condensate. In some embodiments, the condensate is a model for a condensate associated with a disease or illness.

In some embodiments, the condensate is non-naturally occurring. In some embodiments, the condensate is naturally occurring. In some embodiments, the condensate is obtained from a cell source. In some embodiments, the condensate is modified, such as by adding, removing, and/or substituting one or more condensate components.

Examples of types of condensates relevant to the present disclosure include cleavage bodies, P-granules, histone locus bodies, multivesicular bodies, neuronal RNA granules, nuclear gems, nuclear pores, nuclear speckles, nuclear stress bodies, a nucleolus, Oct1/PTF/transcription (OPT) domains, paraspeckles, perinucleolar compartments, PML, nuclear bodies, PML, oncogenic domains, polycomb bodies, processing bodies, signaling clusters, viral condensates, Sam68 nuclear bodies, stress granules, or splicing speckles.

The condensates disclosed herein comprise components, such as macromolecules. In some embodiments, description of a component of the condensate encompasses a molecule associated with, such as in, a condensate. In some embodiments, description of a component of the condensate encompasses a molecule capable of, or known to, associating with a condensate, and said molecule is located in the light phase (e.g., outside the condensate). In some embodiments, the condensate comprises a set of macromolecules, including one or more types of polypeptides (such as one or more polypeptide sequences) and/or one or more types of nucleic acids (such as one or more nucleic acid sequences). In some embodiments, the macromolecule is a polypeptide or a fragment thereof. In some embodiments, the polypeptide or the fragment thereof comprises a Low Complexity Domain or an Intrinsically Disordered Sequence. In some embodiments, the macromolecule comprises a transcription factor or an RNA binding protein. In some embodiments, the macromolecule comprises tau, FUS, huntingtin protein, hnRNPA1, TDP43, PGL-3, or fragments or aggregates thereof. In some embodiments, the macromolecule comprises a nucleic acid, such as RNA or DNA. In some embodiments, the macromolecule is a RNA.

In some embodiments, the component of the condensate is a complex, such as a stable complex, of more than one molecules, such as a macromolecule. In some embodiments, the complex exists in the light phase. In some embodiments, the complex exists in the dense phase.

In some embodiments, the condensate is a homogeneous condensate, e.g., substantially contains a single type of component. In some embodiments, the condensate is heterogeneous, e.g., contains more than one type of component.

In some embodiments, the condensate is a cellular condensate. In some embodiments, the cellular condensate is a cleavage body, a P-granule, a histone locus body, a multivesicular body, a neuronal RNA granule, a nuclear gem, a nuclear pore, a nuclear speckle, a nuclear stress body, a nucleolus, a Oct1/PTF/transcription (OPT) domain, a paraspeckle, a perinucleolar compartment, a PML nuclear body, a PML oncogenic domain, a polycomb body, a processing body, a signaling cluster, a viral condensate, a Sam68 nuclear body, a stress granule, or a splicing speckle.

In some embodiments, the condensate is an extracellular condensate. Extracellular condensates can form in biological solutions outside of a cell, such as the extracellular matrix or plasma, to facilitate reactions or sequester molecules. See, Muiznieks et al., J Mol Biol, 43, 2018, 4741-4753.

The dysregulation of various condensates can be associated with a disease. For example, based on cellular and cell-free condensate experiments, disease-associated mutations in the protein fused in sarcoma (FUS) have been shown to cause aberrant phase-separation behavior that contributes directly to development of the motor neuron disease, amyotrophic lateral sclerosis (ALS). See, Naumann et al., Nat Commun, 9, 2018, 335. In some embodiments, the condensate is a disease-associated condensate. In some embodiments, the disease-associated condensate comprises an alteration, such as compared to a relevant non-disease associated condensate, in one or more of: size of the condensate; shape of the condensate; concentration of one or more components of the condensate; and heterogeneous distribution of components within the condensate, e.g., components located in the core instead of the shell of the condensate.

In some embodiments, the identification of a condensate can be facilitated by the use of a label. Accordingly, in some embodiments, the condensate, such as via a component thereof, comprises a dye or label moiety, such Dendra2, GFP, or RFP. In some embodiments, the dye or labeled compound preferentially partitions in the condensate. In some embodiments, the label is a radioactive label, a colorimetric label, a chemically-reactive label, or a fluorescent label. In some embodiments, the dye or label moiety is associated with, such as conjugated to, a component of the condensate, such as a macromolecule.

### III. Further Aspects Enabled by the Methods Described Herein

In some aspects, the present application provides further aspects that are enabled by the methods described herein. For example, identifying such interactions of a compound, or a portion thereof, and a condensate, or components thereof, enables further use of this information, including intelligent screening and/or design of compounds based on a desired compound activity, a desired partition characteristic, and/or a desired impact the compound has on a condensate phase behavior. In some embodiments, the desired behavior of the compound, or the portion thereof, is based on considerations for modulating disease-associated condensate to alleviate one or more causes or symptoms of the disease.

In some aspects, provided herein is a method of screening for a candidate compound among a plurality of test compounds, or a portion thereof, based on identifying one or more interactions for each compound and a condensate, or a component thereof, using any of the methods described herein. In some embodiments, the candidate compound, or the portion thereof, is selected based on possessing at least one desired interaction with the target condensate, or the component thereof, such as compared to that of a set of screened compounds, or a portion thereof.

In some embodiments, the interaction of a test compound (or a portion thereof) and a target condensate (or a component thereof) is selected from the group consisting of: (i) preferential association of the test compound, or the portion thereof, and the component of the target condensate in the light phase as compared to the dense phase; (ii) preferential association of the test compound, or the portion thereof, and the component of the target condensate in the dense phase as compared to the light phase; (iii) preferential solubility of the test compound, or the portion thereof, in the dense phase of the target condensate as compared to the light phase; (iv) preferential solubility of the test compound, or the portion thereof, in the light phase as compared to the dense phase; (v) preferential association of the test compound, or the portion thereof, and a feature in the dense phase of the target condensate as compared to the light phase; (vi) the test compound, or the portion thereof, competes with a phase-separation driving interaction for the component of the target condensate; (vii) the test compound, or the portion thereof, provides a phase-separation driving interaction for the component of the target condensate; (viii) preferential association of the test compound, or the portion thereof, and another component of the target condensate as compared to the component of the target condensate, which competes with a phase-separation driving interaction for the component of the target condensate; (ix) preferential association of the test compound, or the portion thereof, and another component of the target condensate as compared to the component of the target condensate, which provides a phase-separation driving interaction for the component of the target condensate; (x) preferential association of the test compound, or the portion thereof, at a site not involved in a phase-separation driving interaction as compared to a site involved in a phase-separation driving interaction; and (xi) substantially equal association of the test compound, or the portion thereof, and the component of the condensate in both the light phase and dense phase. In some embodiments, the feature in the dense phase is another component of the condensate. In some embodiments, the feature in the dense phase is a new binding pocket formed in the condensate (or a binding pocket that is found predominantly in the dense phase as compared to the light phase), and includes new binding pockets within the component of the condensate and as formed by interactions of the component of the condensate with another component of the condensate. In some embodiments, the feature in the dense phase is a new configuration of the component of the condensate (or a configuration of the component that is found predominantly in the dense phase as compared to the light phase). In some embodiments, the feature in the dense phase is a favorable microenvironment formed in the condensate.

In some embodiments, the screening method enables comparison of such interactions across a plurality of compounds, thereby enabling the identification of certain moieties (such as chemical moieties) or chemical classes responsible for one or more of such interactions. For example, the screening method can be used to identify a compound, or a portion thereof, that enhances desired property, e.g., a compound, or a portion thereof, that is (i) that strongly partitions in the target condensate; (ii) has a low binding affinity to the component of the condensate in the light phase; and (iii) does not modulate the phase boundary of the component of the condensate. Such candidate compounds can be used for, e.g., preferentially partitioning in the condensate comprising the target component, have a weak or absence of binding to the component of the target condensate in the light phase (i.e., the compound preferentially binds to component in the dense phase compared to the light phase), and does not impact the phase behavior of the condensate.

In some aspects, provided herein is a method of designing a candidate compound having one or more of desired interactions with the target condensate, or the component thereof. In some embodiments, the designing method comprises incorporation of one or more moieties into a candidate compound, wherein each moiety drives a desired interaction. For example, in some embodiments, a candidate compound can be designed having a first moiety that drives partitioning in the condensate, and a second moiety that drives a modulation in a resulting phase behavior of the condensate (e.g., based on modulation of phase boundary characteristic of a component thereof). In some embodiments, the designing method comprises substituting, removing, or adding a moiety associated with one or more of desired compound characteristics and/or drives one or more desired interactions with the target condensate, or the component thereof. In some embodiments, the designing method further comprises obtaining one or more of the compound characteristics described herein for the designed candidate compound, and/or identifying one or more of the interactions of the designed candidate compound with the target condensate (or the component thereof), and determining whether such compound characteristics and/or interactions meet the desired need(s). In some embodiments, the designing method further comprises repeating the designing and testing steps, until the desired need/trait is achieved. In some embodiments, the designing method comprises synthesizing the candidate compound.

In some aspects, provided herein is a method of designing a candidate compound comprising combining two or more moieties, wherein each moiety is associated with one or more desired compound characteristics and/or one or more desired interactions with the target condensate, or the component thereof. In some embodiments, the method of designing comprises attaching a moiety that comprises a desired characteristic identified via the methods described herein at any number of position and/or stereochemical orientations. In some embodiments, the resultant candidate compound comprises the combination of desired compound characteristics and/or one or more desired interactions with the target condensate, or the component thereof. For example, in some embodiments, the candidate compound is designed to contain a first moiety associated with low binding affinity to the component of the condensate in the light phase and strong partitioning characteristic for the condensate, and a second moiety associated with a desired modulation of the phase behavior of the condensate. In some embodiments, the designing method further comprises repeating the designing and testing steps, until the desired need/trait is achieved. In some embodiments, the designing method comprises synthesizing the candidate compound.

In some embodiments, the methods described herein may be used to develop one or more rule sets based on achieved a desired compound characteristic. In some embodiments, the one or more rule sets can be used as a basis for the identification and/or design of one or more compounds using an approach comprising modeling, computer and/or calculation-based techniques, e.g., bioinformatic, cheminformatic, and/or artificial intelligence (AI)-based identification of a compound having a desired partition characteristic. Also provided are computer software for determining and/or applying the one or more rule sets.

In some aspects, provided herein is a library comprising a plurality of compounds, wherein each compound of the plurality of compounds has a feature that drives a desired interaction of the compound, or a portion thereof, and a target condensate and/or a component thereof. In some embodiments, the library comprises a plurality of compounds, wherein each compound of the plurality of compounds comprises a moiety associated with a desired interaction of the moiety and a target condensate and/or a component thereof. In some aspects, provided herein is a library comprising a plurality of compounds, wherein each compound of the plurality of compounds comprises one or more moieties associated with the same (or similar, such as within ±10% activity difference) one or more compound (moiety) characteristics and/or the same (or similar, such as within ±10% activity difference) one or more interactions of the one or more moieties with the target condensate (or the component thereof) as described herein. For example, in some embodiments, there is provided a library comprising a plurality of compounds wherein each compound of the plurality of compounds comprises one or more moieties associated with the same (or similar, such as within ±10% activity difference) binding affinity to a component of a target condensate. For another example, in some embodiments, there is provided a library comprising a plurality of compounds wherein each compound of the plurality of compounds comprises one or more moieties associated with the same (or similar, such as within ±10% activity difference) activity of competing with a phase-separation driving interaction for a component of a target condensate. Such libraries provide compounds with chemical features that correspond to the same (or similar) compound characteristic/interaction, which can facilitate faster and more cost-effective screen.

In some aspects, provided herein is a method of identifying a candidate compound for treating a disease or disorder associated with condensate activity. In some embodiments, the disease or disorder associated with condensate activity refers to a disease or a disorder in which any one or more of the following occurs: 1) a condensate forms; 2) a condensate disappear (e.g., dissolute); 3) a condensate or a component thereof distributes to a location where the condensate or component thereof would not normally locate during healthy condition (e.g., translocate to cytoplasm under disease condition),; 4) condensate number increases or decreases; 5) increase or decrease of the number of condensates comprising and/or not comprising a component (e.g., target component, or one or more other biomolecules that become components of the condensate); 6) a condensate changes size, shape, and/or sphericity; 7) change in condensate composition; 8) change in liquidity (or dynamic) of a condensate; 9) change in solidification of a condensate; 10) presence and/or amount change of fiber formation; 11) change of partitioning of a condensate component into a condensate; and 12) aggregation of a component of a condensate. For example, many neurodegenerative diseases such as ALS and Alzheimer's disease are known to associate with condensate activity.

Based on the one or more of condensate characteristic under the disease condition (and compare to that under a healthy condition), one can identify/screen for/modify/design a candidate compound having a one or more desired compound characteristic(s) and/or desired interaction(s) of the candidate compound with the target condensate (or component thereof), using any of the methods described herein.

For example, for a biomolecule relatively freely diffuses throughout the cytoplasm under healthy condition, but forms/partitions into a condensate under disease condition, possible treatment strategies are: 1) disrupting the condensate to release the biomolecule back into the light (diffuse) phase; 2) excluding the biomolecule from the formed condensate; 3) preventing the biomolecule from partitioning into the condensate; 4) excluding from the condensate a partner biomolecule required to co-form the condensate; and/or 5) preventing a partner biomolecule required to co-form the condensate to interact with the biomolecule for condensate formation, etc. Hence the candidate compound can be identified/screened for/modified/designed to achieve one or more of treatment strategy. For example, a candidate compound is identified/designed with one or more of the desired compound characteristics and/or desired interactions with the condensate (or the component thereof, or other biomolecule related to the condensate formation): (i) strong partition characteristic of the compound to the target condensate; (ii) weak binding affinity to the target biomolecule of the condensate in the light phase; and (iii) strong phase boundary shift for the condensate (or the condensate component), such as excluding the target biomolecule from the formed condensate. Such candidate compound can preferentially binds to the target biomolecule in the dense phase compared to the light phase, thus avoiding off-target activity, and release the target biomolecule from the disease condensate back to where it normally belongs.

### EXEMPLARY EMBODIMENTS

Embodiment 1. A method of identifying one or more interactions of a test compound, or a portion thereof, and a target condensate, or a component thereof, the method comprising obtaining two or more of: (i) a partition characteristic of the test compound, or the portion thereof, for the target condensate; (ii) a binding affinity of the test compound, or the portion thereof, for the component of the target condensate in a light phase; or (iii) a phase boundary shift of the component of the target condensate due to the presence of the test compound, or the portion thereof; and identifying the one or more interactions of the test compound, or the portion thereof, and the target condensate, or the component thereof, based on comparing: (a) the partition characteristic of the test compound, or the portion thereof, for the target condensate; and the binding affinity of the test compound, or the portion thereof, for the component of the target condensate in the light phase, to identify the one or more interactions; (b) the partition characteristic of the test compound, or the portion thereof, for the target condensate; and the phase boundary of the component of the target condensate in the presence of the test compound, or the portion thereof, to identify the one or more interactions; (c) the binding affinity of the test compound, or the portion thereof, for the component of the target condensate in the light phase; and the phase boundary of the component of the target condensate in the presence of the test compound, or the portion thereof, to identify the one or more interactions; or (d) the partition characteristic of the test compound, or the portion thereof, for the target condensate; the binding affinity of the test compound, or the portion thereof, for the component of the target condensate in the light phase; and the phase boundary of the component of the target condensate in the presence of the test compound, or the portion thereof, to identify the one or more interactions.

Embodiment 2. The method of embodiment 1, wherein the interaction of the test compound, or the portion thereof, and the target condensate, or the component thereof, is selected from the group consisting of: (i) preferential association of the test compound, or the portion thereof, and the component of the target condensate in the light phase as compared to the dense phase; (ii) preferential association of the test compound, or the portion thereof, and the component of the target condensate in the dense phase as compared to the light phase; (iii) preferential solubility of the test compound, or the portion thereof, in the dense phase of the target condensate as compared to the light phase; (iv) preferential solubility of the test compound, or the portion thereof, in the light phase as compared to the dense phase; (v) preferential association of the test compound, or the portion thereof, and a feature in the dense phase of the target condensate as compared to the light phase; (vi) the test compound, or the portion thereof, competes with a phase-separation driving interaction for the component of the target condensate; (vii) the test compound, or the portion thereof, provides a phase-separation driving interaction for the component of the target condensate; (viii) preferential association of the test compound, or the portion thereof, and another component of the target condensate as compared to the component of the target condensate, which competes with a phase-separation driving interaction for the component of the target condensate; (ix) preferential association of the test compound, or the portion thereof, and another component of the target condensate as compared to the component of the target condensate, which provides a phase-separation driving interaction for the component of the target condensate; (x) preferential association of the test compound, or the portion thereof, at a site not involved in a phase-separation driving interaction as compared to a site involved in a phase-separation driving interaction; and (xi) substantially equal association of the test compound, or the portion thereof, and the component of the condensate in both the light phase and dense phase.

Embodiment 3. The method of embodiment 1 or 2, wherein the partition characteristic of the test compound, or the portion thereof, for the target condensate comprises the presence or absence of partitioning of the test compound, or the portion thereof, in the target condensate.

Embodiment 4. The method of embodiment 3, wherein the presence or absence of partitioning of the test compound, or the portion thereof, in the target condensate is determined based on a partition characteristic threshold value.

Embodiment 5. The method of embodiment 4, wherein the presence of partitioning of the test compound, or the portion thereof, in the target condensate is determined based on having the partition characteristic threshold value of 1 or more.

Embodiment 6. The method of any one of embodiments 1-5, wherein the partition characteristic of the test compound, or the portion thereof, for the target condensate comprises the degree of partitioning of the test compound, or the portion thereof, in the target condensate.

Embodiment 7. The method of any one of embodiments 1-6, wherein the binding affinity of the test compound, or the portion thereof, for the component of the target condensate in the light phase comprises the presence or absence of a binding association of the test compound, or the portion thereof, and the component of the target condensate.

Embodiment 8. The method of embodiment 7, wherein the presence or absence of the binding association is determined based on a binding affinity threshold value.

Embodiment 9. The method of embodiment 8, wherein the presence of the binding association of the test compound, or the portion thereof, and the component of the target condensate is determined based on having the binding affinity threshold value of 10 mM or less.

Embodiment 10. The method of any one of embodiments 1-9, wherein the binding affinity of the test compound, or the portion thereof, for the component of the target condensate comprises the degree of the binding association of the test compound, or the portion thereof, and the component of the target condensate.

Embodiment 11. The method of any one of embodiments 1-10, wherein the phase boundary shift of the component of the target condensate due to the presence of the test compound, or the portion thereof comprises the presence or absence of a phase behavior.

Embodiment 12. The method of any one of embodiments 1-11, wherein identifying the one or more interactions of the test compound, or the portion thereof, and the target condensate, or the component thereof, based on comparing one of (a)-(d) further comprises comparing to a reference.

Embodiment 13. The method of embodiment 12, wherein the reference comprises information obtained using a reference compound regarding one or more of a partition characteristic of the reference compound for the target condensate, a binding affinity of the reference compound for the component of the condensate in the light phase, and a phase boundary shift of the component of the target condensate due to the presence of the reference compound.

Embodiment 14. The method of embodiment 12 or 13, wherein the reference comprises information obtained using a plurality of reference compounds.

Embodiment 15. The method of embodiment 14, wherein the plurality of reference compounds comprises compounds in the same chemical class as the test compound.

Embodiment 16. The method of embodiment 14 or 15, wherein the plurality of reference compounds comprises compounds in different chemical classes as the test compound.

Embodiment 17. The method of any one of embodiments 14-16, wherein the plurality of reference compounds comprises at least 5 compounds.

Embodiment 18. The method of any one of embodiments 1-17, further comprising obtaining a mode of binding for the test compound and the component of the target condensate.

Embodiment 19. The method of embodiment 18, wherein the mode of binding is determined via a polyphasic linkage formalism technique.

Embodiment 20. The method of any one of embodiments 1-19, further comprising measuring the partition characteristic of the test compound, or the portion thereof, for the target condensate.

Embodiment 21. The method of embodiment 20, wherein measuring the partition characteristic of the test compound, or the portion thereof, for the target condensate comprises measuring the amount of the test compound, or the portion thereof, in the target condensate.

Embodiment 22. The method of embodiment 21, wherein measuring the amount of the test compound, or the portion thereof, in the target condensate is determined via measuring the amount of the test compound, or the portion thereof, in an extra-condensate solution.

Embodiment 23. The method of any one of embodiments 20-22, wherein the partition characteristic of the test compound, or the portion thereof, for the target condensate is measured using a confocal microscopy or fluorescence spectroscopy technique.

Embodiment 24. The method of any one of embodiments 20-22, wherein the partition characteristic of the test compound, or the portion thereof, for the target condensate is measured by: (a) combining the test compound and a composition comprising the target condensate and an extra-condensate solution; (b) obtaining a reference control; (c) measuring an MS signal of the test compound in the extra-condensate solution, or a portion thereof, using a mass spectrometry technique; (d) measuring an MS signal of the test compound in the reference control, or a portion thereof, using a mass spectrometry technique; and (e) comparing the MS signal of the test compound from the extra-condensate solution and the MS signal of the test compound from the reference control.

Embodiment 25. The method of any one of embodiments 1-24, further comprising measuring the binding affinity of the test compound, or the portion thereof, for the component of the target condensate in a light phase.

Embodiment 26. The method of embodiment 25, wherein obtaining the binding affinity of the test compound, or the portion thereof, for the component of the condensate in the light phase comprises measuring the dissociation constant (K_{d}) of the test compound, or the portion thereof, for the component of the condensate in the light phase.

Embodiment 27. The method of embodiment 25 or 26, wherein measuring the binding affinity of the test compound, or the portion thereof, for the component of the condensate in the light phase comprises a MicroScale Thermophoresis (MST), isothermal titration calorimetry (ITC), surface plasmon resonance (SPR), nuclear magnetic resonance (NMR), or fluorescence polarization (FP) technique.

Embodiment 28. The method of any one of embodiments 1-27, further comprising measuring the phase boundary shift of the component of the target condensate due to the presence of the test compound, or the portion thereof.

Embodiment 29. The method any one of embodiments 28, wherein the phase boundary shift is the partition characteristic of the component of the target condensate for the target condensate.

Embodiment 30. The method of embodiment 28 or 29, wherein the phase boundary shift of the component of the target condensate due to the presence of the test compound, or the portion thereof, is measured using a microscopy, fluorescence spectroscopy, ultraviolet-visible (UV-Vis) spectroscopy, fluorescence recovery after photobleaching (FRAP), Static and Dynamic Light Scattering (SLS/DLS), or mass spectrometry-based technique.

Embodiment 31. The method of any one of embodiments 1-30, wherein the component of the target condensate is a macromolecule.

Embodiment 32. The method of any one of embodiments 1-31, wherein the component of the target condensate comprises a polypeptide.

Embodiment 33. The method of any one of embodiments 1-32, wherein the component of the target condensate comprises a nucleic acid.

Embodiment 34. The method of any one of embodiments 1-33, further comprising determining one or more contributing factors associated with a partition characteristic of the test compound, or the portion thereof, for a reference condensate.

Embodiment 35. The method of embodiment 34, further comprising comparing the one or more contributing factors associated with the partition characteristic of the test compound, or the portion thereof, for the target condensate with the one or more contributing factors associated with the partition characteristic of the test compound, or the portion thereof, for the reference condensate.

Embodiment 36. A library comprising a plurality of compounds, wherein each compound of the plurality of compounds comprises a moiety associated with a desired interaction of the moiety and a target condensate and/or a component thereof.

Embodiment 37. A method of designing a compound having one or more desired interactions with a target condensate, or a component thereof, the method comprising: (a) identifying one or more interactions of a candidate compound, or a portion thereof, and the target condensate, or the component thereof according to any one of embodiments 1-35; and (b) designing the compound based on the candidate compound, or the portion thereof associated with the identified one or more interactions.

Embodiment 38. A method of designing a test compound having a desired interaction profile, the method comprising modifying a precursor of the test compound by attaching a moiety to the compound, wherein the moiety comprises a characteristic having one or more desired interactions with a target condensate, or a component thereof.

Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### EXAMPLES

### Example 1

This example illustrates exemplary measurements from an analysis of Rhodamine B (RhoB) and a condensate comprising a FUS component using the methods described herein. The binding affinity of RhoB to FUS protein in the light phase, the partition characteristic of RhoB to FUS condensate, and RhoB's ability to modulate FUS condensate were examined.

SNAP-tagged FUS protein (“FUS-SNAP”) and RhoB were mixed and subjected to a phase separation condition. The final concentration of RhoB in the reaction was 5 μM and 1.25 μM. For control experiment, no RhoB was added. The partition characteristic of RhoB to FUS-SNAP condensate was then measured by both a fluorescence spectroscopy and a mass spectroscopy-based technique described herein.

Images of the condensate droplets were acquired with a confocal microscope for appropriate setting for RhoB. The background signal of FUS-SNAP droplets in the absence of RhoB was also recorded. Fluorescence intensity was measured inside a FUS-SNAP condensate and in a region next to (outside) the FUS-SNAP condensate, and corrected for background signal (no RhoB controls), to calculate the fraction of RhoB in extra-condensate solution (i.e., supernatant).

A mass spectrometry-based method was used to measure the partition characteristics of RhoB and compared to measurements obtained using the fluorescence-based assay above. RhoB partitioning into the FUS-SNAP condensate will cause a reduction of the concentration outside (RhoB in the extra-condensate solution or light phase) and an increase of the concentration inside (RhoB in the FUS-SNAP droplets). After phase separation incubation, reactions were processed to separate condensates from the supernatant (the light phase) by centrifugation. The amount of RhoB in the supernatant and a reference reaction were measured using mass spectrometry. The partition characteristic was then calculated based on the fraction of RhoB in extra-condensate solution (i.e., supernatant).

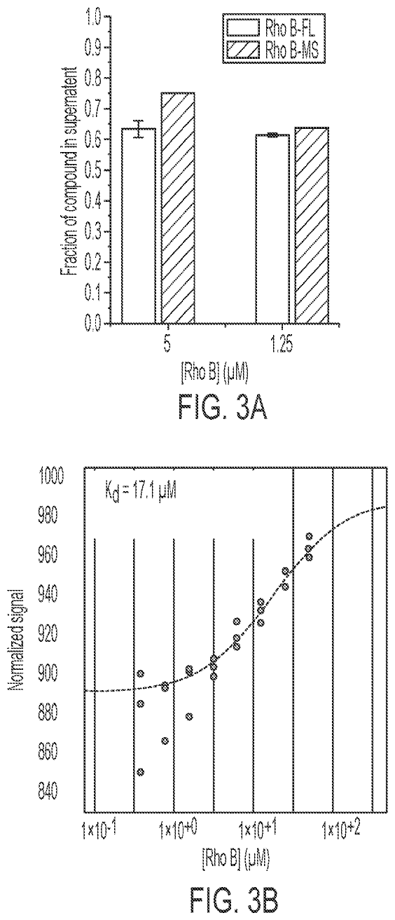

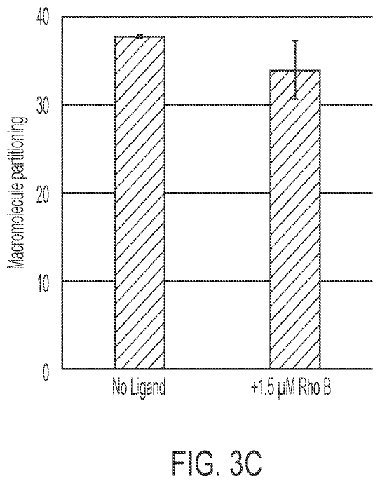

As shown in FIG. 3A, both fluorescence spectroscopy (RhoB-FL) and mass spectroscopy (RhoB-MS) analysis demonstrated that RhoB (under both concentrations) partitions into FUS-SNAP condensates formed in vitro, as reflected by the fraction of RhoB in the light phase extra-condensate solution (supernatant). The two measurement types are in agreement.

The binding affinity (K_{d}) of RhoB to FUS-SNAP protein in the light phase was measured using Dianthus MST technology. As can be seen from FIG. 3B, RhoB binds to FUS-SNAP protein in the light phase with a K_{d} of 17.1 μM.

EGFP-tagged FUS protein (“FUS-EGFP”) and RhoB were mixed and subjected to phase separation condition. The final concentration of RhoB in the reaction was 1.5 μM. For control experiment, no RhoB was added. Images were acquired with a confocal microscope, and EGFP signal inside (I-in) and outside (I-out) of the FUS-EGFP condensate was measured as described above.

As can be seen from FIG. 3C, EGFP-labeled FUS partitioned less into the condensate in the presence (1.5 μM) of RhoB, indicating that RhoB has certain phase modulating activity for FUS condensates.

Based on these measurements, information regarding the one or more interactions of RhoB and the FUS condensate, or the compound thereof—namely FUS, can be further extracted. For example, the presence of binding affinity between RhoB and FUS indicates this as a possible driving force for the observed partitioning of RhoB in FUS condensates.

### Example 2

This example demonstrates the identification of interactions of a compound (Rho800) and a condensate, or a component thereof, using the methods described herein. Specifically, provided is a titration experiment of Rho800 in the presence of a FUS-containing condensate (FUS-mEGFP/RNA droplets), and the resulting impact on partitioning of Rho800 and FUS. Further provided are binding experiments involving FUS and Rho800.

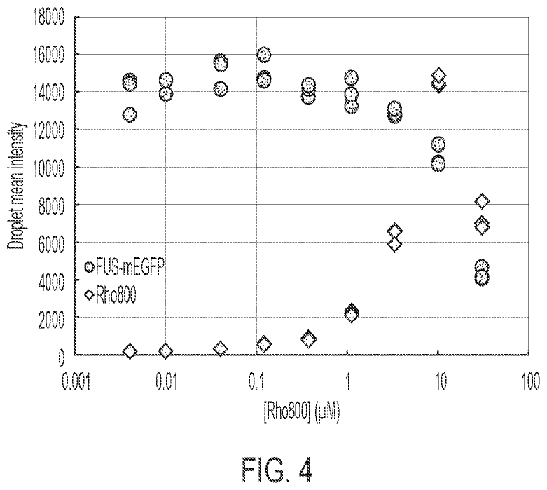

For partitioning study, droplets were prepared in a low-attachment 384-well plate by mixing recombinant FUS-mEGFP protein with synthetic PrD RNA, in the presence of the indicated concentration of Rho800, at a final concentration of 1 mM FUS-mEGFP and 250 nM PrD RNA, using an automated liquid handler. The droplets were incubated at room temperature, in the dark for 4 hours and imaged using a high content confocal fluorescence microscope. Values indicated on the y-axis of FIG. 4 (n=3) were obtained by quantitative image analysis and represent the mean per well intensity of FUS-mEGFP and Rho800 inside segmented droplets.

For binding study, recombinant ¹⁵N-isotope and ¹⁵N/¹³C-labeled FUS-RRM proteins were expressed in E. coli and purified using published methods. NMR peak assignments were transferred from publicly available data repositories and confirmed using standard 3D NMR methods. 100 mM protein was titrated with Rho800, ¹H/¹⁵N-HSQC (heteronuclear single quantum coherence) were recorded, and chemical shift perturbations (CSPs) were calculated from the ±180 mM Rho800 spectra.

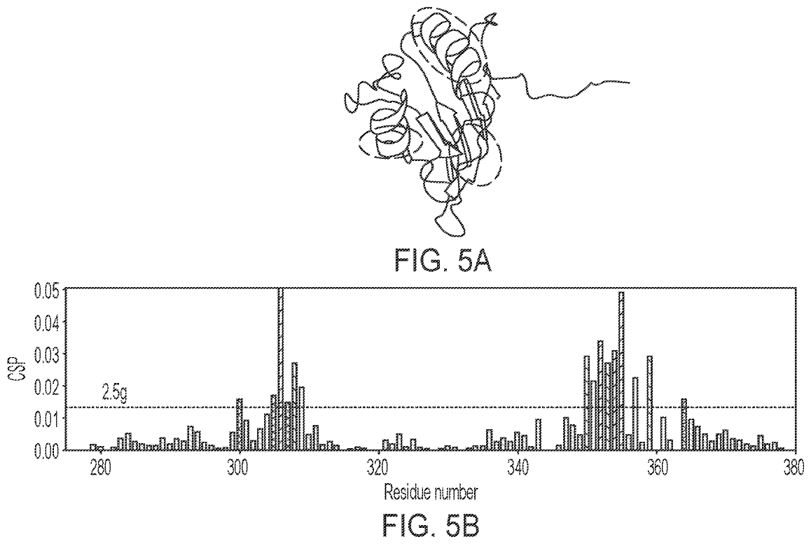

As illustrated in FIG. 4, increasing concentrations of Rho800 increased partitioning of the compound into the condensate droplets. In contrast, increasing concentrations of Rho800 resulting in FUS-mEGFP de-partitioning from the condensate droplets. Once FUS-mEGFR in the condensate drops below a certain level, partitioning of Rho800 into the condensate also decreases (compare data points at the highest Rho800 concentration with others in FIG. 4). As illustrated in FIG. 5B, CSPs obtained by ¹H/¹⁵N-HSQC 2D-NMR mapped the binding site of Rho800 onto FUS-RRM domain. The affinity of Rho800 for FUS-RRM was estimated to be about 50 mM to about 500 mM. The FUS residues involved in Rho800 binding are further illustrated in FIG. 5A (in dashed circles). This data shows that Rho800 binds directly to the FUS-RRM domain. Thus, it can be concluded in view of the partitioning data that direct binding is one of the driving forces for Rho800 partitioning. The less partitioning of FUS-mEGFP into the condensate in the presence of increasing amount of Rho800 indicates that Rho800 has certain phase modulating activity for FUS condensates, such as by directly binding to FUS in the light phase to prevent FUS's partitioning into the condensate, and/or by shifting the phase boundary of FUS so more FUS is required to phase separate to form a condensate at the same phase separation control parameter in the presence of Rho800.

### Example 3

This example demonstrates the use of the methods described herein to identify one or more interactions of four compounds for a FUS-containing condensate, or a component thereof, thus identifying more granular information regarding driving forces involved in small molecule partitioning (or absence thereof).

Partitioning was measured according to previous Examples, using a confocal fluorescence microscope. Binding association (K_{d}) was measured using MST. Classifications of characteristics are as follows. Binding affinity characteristic: Strong (++) (K_{d}<10 μM); (+) Medium (10 μM≤K_{d}<100 μM); (−) Weak (K_{d}≥100 μM). Partitioning characteristic: (++) Strong (PC≥100); (+) Medium (100>PC≥10); (−) Weak (PC<10). Phase boundary characteristic: (++) Strong (EC50<10 μM); (+) Medium (10 μM≤EC50<100 μM); (−) Weak (EC50≥100 μM).

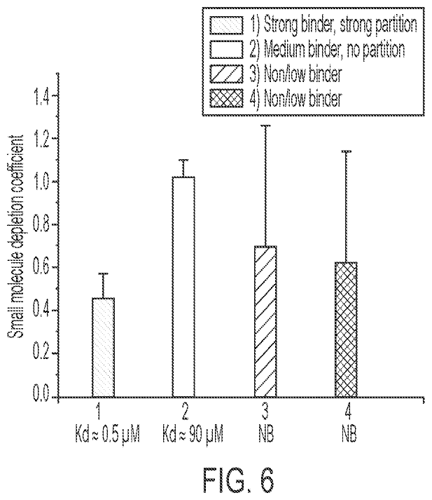

As shown in FIG. 6, compound 1 strongly partitions into the condensate, and compound 1 also strongly binds a component of the condensate in the light phase. Additionally, it was observed that compound 1 did not have an effect on a phase boundary characteristic of the component of the condensate. Thus, direct binding of compound 1 is a driving force of partitioning, and presence of compound 1 does not impact a phase boundary characteristic of the component of the condensate.

As shown in FIG. 6, compound 2 does not partition into the condensate, and compound 2 is a medium binder of a component of the condensate in the light phase. Additionally, it was observed that compound 2 did not have an effect on a phase boundary characteristic of the component of the condensate. Thus, medium binding association of compound 2 is not a driving force of partitioning, and presence of compound 2 does not impact a phase boundary characteristic of the component of the condensate.

As shown in FIG. 6, compound 3 partitions into the condensate, and compound 3 does not bind (ND; not detected) a component of the condensate in the light phase. Additionally, it was observed that compound 3 had a weak effect on a phase boundary characteristic of the component of the condensate. Thus, binding association (or lack thereof) is not involved as a driving force of partitioning.

As shown in FIG. 6, compound 4 partitions into the condensate, and compound 4 does not bind (ND; not detected) a component of the condensate in the light phase. Additionally, it was observed that compound 4 had a no effect on a phase boundary characteristic of the component of the condensate. Thus, binding association (or lack thereof) is not involved as a driving force of partitioning.

The results discussed above are presented in Table 1.

TABLE 1
Summary table of results.

| | Condensate partition | Direct binding | Phase boundary |
|---|---|---|---|
| Compound | characteristic | characteristic | characteristic |
| 1 | ++ | ++ | No effect |
| 2 | − | + | No effect |
| 3 | + | − | Weak effect |
| 4 | + | − | No effect |

What is claimed is:

1. A method of identifying one or more interactions of a test compound, or a portion thereof, and a target condensate, or a component thereof, the method comprising:
obtaining two or more of:
(i) a partition characteristic of the test compound, or the portion thereof, for the target condensate;
(ii) a binding affinity characteristic of the test compound, or the portion thereof, for the component of the target condensate in a light phase; or
(iii) a phase boundary characteristic of the component of the target condensate in the presence of the test compound, or the portion thereof; and
identifying the one or more interactions of the test compound, or the portion thereof, and the target condensate, or the component thereof, based on comparing two or more of (i), (ii), and (iii) to identify the one or more interactions.

2. The method of claim 1, wherein identifying the one or more interactions of the test compound, or the portion thereof, and the target condensate, or the component thereof, is based on comparing the partition characteristic of the test compound, or the portion thereof, for the target condensate; and the binding affinity characteristic of the test compound, or the portion thereof, for the component of the target condensate in the light phase.

3. The method of claim 1, wherein identifying the one or more interactions of the test compound, or the portion thereof, and the target condensate, or the component thereof, is based on comparing the partition characteristic of the test compound, or the portion thereof, for the target condensate; and the phase boundary characteristic of the component of the target condensate in the presence of the test compound, or the portion thereof.

4. The method of claim 1, wherein identifying the one or more interactions of the test compound, or the portion thereof, and the target condensate, or the component thereof, is based on comparing the binding affinity characteristic of the test compound, or the portion thereof, for the component of the target condensate in the light phase; and the phase boundary characteristic of the component of the target condensate in the presence of the test compound, or the portion thereof.

5. The method of claim 1, wherein identifying the one or more interactions of the test compound, or the portion thereof, and the target condensate, or the component thereof, is based on comparing the partition characteristic of the test compound, or the portion thereof, for the target condensate; the binding affinity characteristic of the test compound, or the portion thereof, for the component of the target condensate in the light phase; and the phase boundary characteristic of the component of the target condensate in the presence of the test compound, or the portion thereof.

6. The method of any one of claims 1-5, wherein the interaction of the test compound, or the portion thereof, and the target condensate, or the component thereof, is selected from the group consisting of:
(1) a preferential association of the test compound, or the portion thereof, and the component of the target condensate in the light phase as compared to a dense phase;
(2) a preferential association of the test compound, or the portion thereof, and the component of the target condensate in the dense phase as compared to the light phase;
(3) a preferential solubility of the test compound, or the portion thereof, in the dense phase of the target condensate as compared to the light phase;
(4) a preferential solubility of the test compound, or the portion thereof, in the light phase as compared to the dense phase;
(5) a preferential association of the test compound, or the portion thereof, and a feature in the dense phase of the target condensate as compared to the light phase;
(6) an ability of the test compound, or the portion thereof, to compete with a phase-separation driving interaction for the component of the target condensate;
(7) an ability of the test compound, or the portion thereof, to provide a phase-separation driving interaction for the component of the target condensate;
(8) a preferential association of the test compound, or the portion thereof, and another component of the target condensate as compared to the component of the target condensate, wherein the preferential association of the test compound, or the portion thereof, and the other component of the target condensate hinders a phase-separation driving interaction for the component of the target condensate;
(9) a preferential association of the test compound, or the portion thereof, and another component of the target condensate as compared to the component of the target condensate, wherein the preferential association of the test compound, or the portion thereof, and the other component of the target condensate provides a phase-separation driving interaction for the component of the target condensate;
(10) a preferential association of the test compound, or the portion thereof, at a site of the component not involved in a phase-separation driving interaction as compared to a site of the component involved in a phase-separation driving interaction; and
(11) a substantially equal association of the test compound, or the portion thereof, and the component of the condensate in both the light phase and the dense phase.

7. The method of any one of claims 1-6, wherein the partition characteristic of the test compound, or the portion thereof, for the target condensate indicates the presence or absence of partitioning of the test compound, or the portion thereof, in the target condensate.

8. The method of claim 7, wherein the presence or absence of partitioning of the test compound, or the portion thereof, in the target condensate is determined based on a partition characteristic threshold value.

9. The method of claim 8, wherein the presence of partitioning of the test compound, or the portion thereof, in the target condensate is determined based on having the partition characteristic of more than 1.

10. The method of any one of claims 1-9, wherein the partition characteristic of the test compound, or the portion thereof, for the target condensate indicates the degree of partitioning of the test compound, or the portion thereof, in the target condensate.

11. The method of any one of claims 1-10, wherein the partition characteristic of the test compound, or the portion thereof, for the target condensate is based on a ratio of the test compound, or the portion thereof, in the dense phase of the target condensate versus the test compound, or the portion thereof, in the light phase.

12. The method of any one of claims 1-11, wherein the binding affinity characteristic of the test compound, or the portion thereof, for the component of the target condensate in the light phase indicates the presence or absence of a binding association of the test compound, or the portion thereof, and the component of the target condensate in the light phase.

13. The method of claim 12, wherein the presence or absence of the binding association is determined based on a binding affinity threshold value.

14. The method of claim 13, wherein the presence of the binding association of the test compound, or the portion thereof, and the component of the target condensate in the light phase is determined based on having the binding affinity of about 10 mM or less.

15. The method of any one of claims 1-14, wherein the binding affinity characteristic of the test compound, or the portion thereof, for the component of the target condensate in the light phase indicates the degree of the binding association of the test compound, or the portion thereof, and the component of the target condensate in the light phase.

16. The method of any one of claims 1-15, wherein the binding affinity characteristic of the test compound, or the portion thereof, for the component of the target condensate in the light phase is based on a dissociation constant (K_{d}) of the test compound, or the portion thereof, for the component of the target condensate in the light phase.

17. The method of any one of claims 1-16, wherein the phase boundary characteristic of the component of the target condensate indicates the presence or absence of modulated partitioning of the component of the target condensate for the target condensate due to the presence of the test compound, or the portion thereof.

18. The method of any one of claims 1-17, wherein the phase boundary characteristic is based on a phase diagram.

19. The method of any one of claims 1-18, wherein identifying the one or more interactions of the test compound, or the portion thereof, and the target condensate, or the component thereof, based on comparing two or more of (i), (ii), and (iii), further comprises comparing to a reference.

20. The method of claim 19, wherein the reference comprises information obtained using a reference compound regarding one or more of a partition characteristic of the reference compound for the target condensate, a binding affinity characteristic of the reference compound for the component of the target condensate in the light phase, and a phase boundary characteristic of the component of the target condensate in the presence of the reference compound.

21. The method of claim 19 or 20, wherein the reference comprises information obtained using a plurality of reference compounds.

22. The method of claim 21, wherein the plurality of reference compounds comprises compounds in the same chemical class as the test compound.

23. The method of claim 21, wherein the plurality of reference compounds comprises compounds in different chemical classes as the test compound.

24. The method of any one of claims 21-23, wherein the plurality of reference compounds comprises at least 5 reference compounds.

25. The method of any one of claims 1-24, further comprising obtaining a mode of binding for the test compound and the component of the target condensate.

26. The method of claim 25, wherein the mode of binding is determined via a polyphasic linkage formalism technique.

27. The method of any one of claims 1-26, further comprising measuring the partition characteristic of the test compound, or the portion thereof, for the target condensate.

28. The method of claim 27, wherein measuring the partition characteristic of the test compound, or the portion thereof, for the target condensate comprises measuring the amount of the test compound, or the portion thereof, in the target condensate.

29. The method of claim 28, wherein measuring the amount of the test compound, or the portion thereof, in the target condensate is determined via measuring the amount of the test compound, or the portion thereof, in an extra-condensate solution.

30. The method of any one of claims 27-29, wherein the partition characteristic of the test compound, or the portion thereof, for the target condensate is measured using a confocal microscopy or fluorescence spectroscopy technique.

31. The method of any one of claims 27-30, wherein the partition characteristic of the test compound, or the portion thereof, for the target condensate is measured by:
(a) combining the test compound and a composition comprising or subjected to forming the target condensate and an extra-condensate solution;
(b) obtaining a reference control;
(c) measuring a mass spectrometry (MS) signal of the test compound in the extra-condensate solution, or a portion thereof, using an MS technique;
(d) measuring an MS signal of the test compound in the reference control, or a portion thereof, using an MS technique; and
(e) comparing the MS signal of the test compound from the extra-condensate solution and the MS signal of the test compound from the reference control.

32. The method of any one of claims 1-31, further comprising measuring the binding affinity characteristic of the test compound, or the portion thereof, for the component of the target condensate in the light phase.

33. The method of claim 32, wherein measuring the binding affinity characteristic of the test compound, or the portion thereof, for the component of the condensate in the light phase comprises measuring the dissociation constant (K_{d}) of the test compound, or the portion thereof, for the component of the condensate in the light phase.

34. The method of claim 32 or 33, wherein measuring the binding affinity characteristic of the test compound, or the portion thereof, for the component of the condensate in the light phase comprises using a MicroScale Thermophoresis (MST), isothermal titration calorimetry (ITC), surface plasmon resonance (SPR), nuclear magnetic resonance (NMR), or fluorescence polarization (FP) technique.

35. The method of any one of claims 1-34, further comprising measuring the phase boundary characteristic of the component of the target condensate due to the presence of the test compound, or the portion thereof.

36. The method any one of claims 1-35, wherein the phase boundary characteristic is representative of a partition characteristic of the component of the target condensate for the target condensate.

37. The method of claim 35 or 36, wherein the phase boundary characteristic of the component of the target condensate in the presence of the test compound, or the portion thereof, is measured using a microscopy, fluorescence spectroscopy, ultraviolet—visible (UV-Vis) spectroscopy, fluorescence recovery after photobleaching (FRAP), Static and Dynamic Light Scattering (SLS/DLS), or mass spectrometry-based technique.

38. The method of any one of claims 1-37, wherein the component of the target condensate is a macromolecule.

39. The method of any one of claims 1-38, wherein the component of the target condensate comprises a polypeptide.

40. The method of any one of claims 1-39, wherein the component of the target condensate comprises a nucleic acid.

41. The method of any one of claims 1-40, further comprising determining one or more contributing factors associated with a partition characteristic of the test compound, or the portion thereof, for a reference condensate.

42. The method of claim 41, further comprising comparing the one or more contributing factors associated with the partition characteristic of the test compound, or the portion thereof, for the target condensate with the one or more contributing factors associated with the partition characteristic of the test compound, or the portion thereof, for the reference condensate.

43. A method of designing a compound having one or more desired interactions with a target condensate, or a component thereof, the method comprising:
(a) identifying one or more interactions of a candidate compound, or a portion thereof, and the target condensate, or the component thereof, according to the method of any one of claims 1-42; and
(b) designing the compound based on the candidate compound, or the portion thereof, associated with the identified one or more interactions.

44. A method of designing a compound having a desired interaction profile, the method comprising modifying a precursor of the compound by attaching a moiety to the precursor, wherein the moiety comprises a characteristic having one or more desired interactions with a target condensate, or a component thereof, identified according to the method of any one of claims 1-42.
